(19) United States
(12) Patent Application Publication
(10) Pub. No.: US 2026/0256849 A1
(43) Pub. Date: Sep. 3, 2026

(54) METHODS AND COMPOSITIONS FOR ENRICHMENT OF TARGET CELLS

(71) Applicant: PLURI BIOTECH LTD., Haifa (IL)

(72) Inventors: Rachel OFIR, Adi (IL); Zami ABERMAN, Tel-Mond (IL); Niva SHRAGA HELED, Nahariya (IL)

(21) Appl. No.: 19/657,719

(22) Filed: Apr. 24, 2026

Related U.S. Application Data
(62) Division of application No. 17/923,431, filed on May 5, 2021, now abandoned.
(60) Provisional application No. 63/020,027, filed on May 5, 2020.

(51) Int. Cl. A61K 35/50; C12N 5/0775

(52) U.S. Cl. CPC A61K 35/50; C12N 5/0668; C12N 2500/90

(57) ABSTRACT

Disclosed herein are compositions comprising subcellular fractions from expanded placental adherent stromal cells, and methods utilizing same.

### CROSS REFERENCE TO RELATED APPLICATIONS

This application is a divisional application of U.S. patent application Ser. No. 17/923,431, filed Nov. 4, 2022, which is a National Phase of PCT Application No. PCT/IL2021/050514 having International filing date of May 5, 2021, which claims the benefit of priority of U.S. Provisional Patent Application No. 63/020,027, filed May 5, 2020, the contents of which are all incorporated herein by reference in their entirety.

### FIELD

Disclosed herein are methods and compositions for enrichment of target cells, comprising subcellular fractions from adherent stromal cells.

### BACKGROUND

Cells are enveloped by a plasma membrane that regulates movement of substances in and out of the cells and maintains its electric potential. This membrane is composed primarily of a double layer of phospholipids, which are amphiphilic. Embedded within this membrane are a variety of protein molecules. Inside the membrane, the cytoplasm makes up a significant portion of the cell's volume.

Organelles are parts of the cell which are adapted and/or specialized for carrying out one or more vital functions. Many such organelles are encapsulated by their own membranes. Vesicular components include both intracellular compartments, e.g., mitochondria, endoplasmic reticulum, Golgi apparatus, lysosomes, endosomes, and peroxisomes; and secreted components, e.g., exosomes, microvesicles, exomeres, ectosomes, and microparticles.

Clark and Shay (1982) discovered that mitochondria with antibiotic-resistant genes could be transferred to antibiotic-sensitive cells, enabling their survival in selective medium. Since then, research has been performed on developing techniques for artificial mitochondrial transfer. Techniques can be broadly categorized into coculture of the donor and recipient cells, and incubation of recipient cells with purified mitochondria or mitochondria-enriched preparations (Caicedo A et al., 2017). Mitochondrial enrichment has potential utility in treating cachexia (Boengler K et al.), muscular dystrophies, (Kennedy T L et al.), and mitochondrial myopathies (Saha P P et al.), all of which represent major unmet medical needs.

### SUMMARY

Provided herein are methods and compositions for enhancing function of target cells, and therapeutic modalities, comprising sub-cellular fractions of placental adherent stromal cells (ASC).

In some embodiments, there is provided a pharmaceutical composition for enhancing a cellular function, comprising a therapeutically effective amount of a subcellular fraction of placental ASC. In certain embodiments, the cellular function is cellular aerobic respiration. Methods of monitoring cellular aerobic respiration are known in the art; non-limiting examples of which are described, for example, in Mito T, et al.

In another embodiment, there is provided use of mitochondria of placental ASC in the preparation of a medicament for enhancing cellular function.

In certain embodiments, the placental ASC that are the source of the subcellular fractions have been cultured on a 2-dimensional (2D) substrate, a 3-dimensional (3D) substrate, or a combination thereof. Non-limiting examples of 2D and 3D culture conditions are provided in the Detailed Description and in the Examples. Alternatively or in addition, the placental ASC are maternal tissue-derived ASC, fetal tissue-derived ASC, or a mixture thereof.

Reference herein to “growth” of a population of cells is intended to be synonymous with expansion of a cell population. In certain embodiments, ASC (which are, in some embodiments, placental ASC), are expanded without substantial differentiation. In various embodiments, the described expansion is on a 2D substrate; on a 3D substrate; or on a 2D substrate, followed by a 3D substrate. Except where otherwise indicated, all ranges mentioned herein are inclusive.

As used herein the phrase “adherent cells” refers to cells capable of attaching to an attachment substrate and expanding or proliferating on the substrate. In some embodiments, the cells are anchorage dependent, i.e., require attachment to a surface to proliferate and grow in vitro.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2 are cryo-TEM images of vesicle preparations (see Example 5)

FIG. 3 shows the results of KEGG analysis on proteins identified in placental cell exosomes preparation.

FIG. 4 is a chart of siRNA identified in placental exosome preparations, together with Uniprot codes of a non-exhaustive/representative list of proteins inhibited by each miRNA

FIGS. 5A-B are schematic diagrams of an experimental protocol for measuring mitochondria dysfunction by MitoSox/CyQuant staining, followed by fluorescence measurement without (A) or with (B) treatment with ASC or CM from ASC,

FIG. 6 depicts the CHBP structure.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise subcellular fractions of placental adherent stromal cells (ASC), e.g., cultured placental ASC. In some embodiments, the ASC may be human ASC, or in other embodiments animal ASC. Alternatively or in addition, the subcellular fraction comprises mitochondria, or, in other embodiments, is enriched in mitochondria. In other embodiments, the subcellular fraction comprises exosomes, or, in other embodiments, is enriched in exosomes. In still other embodiments, the subcellular fraction comprises other organelles, or, in other embodiments, is enriched in other organelles (e.g., organelles described herein), each of which is considered a separate embodiment. In yet other embodiments, the subcellular fraction is a whole cell lysate. In other embodiments, the subcellular fraction is a whole cell lysate that has been depleted of nuclei.

Methods for cell fractionation are known in the art. Non-limiting examples of such methods are described in Mater Methods 2013; 3:562 by Parisis Nikos.

In some embodiments, there is provided a method of enhancing a cellular function in a subject, comprising the step of administering to the subject a pharmaceutical composition comprising a subcellular fraction of placental ASC, thereby enhancing a cellular function. In more specific embodiments, the placental ASC are from the same species as the subject. In other embodiments, the placental ASC are xenogenic to the subject.

The described subcellular fraction is, in some embodiments, a vesicular component of a placental ASC, which may be, in more specific embodiments, an intracellular compartment, or in other embodiments, a secreted component.

In other embodiments, the vesicular component is enriched in mitochondria; or in other embodiments, endoplasmic reticulum; or in other embodiments, Golgi apparatus; or, in other embodiments, lysosomes; or, in other embodiments, peroxisomes. In still other embodiments, the vesicular component consists essentially of mitochondria; or in other embodiments, endoplasmic reticulum; or in other embodiments, Golgi apparatus; or, in other embodiments, lysosomes; or, in other embodiments; peroxisomes, or in, other embodiments, endosomes. In still other embodiments, the vesicular component consists of mitochondria; or in other embodiments, endoplasmic reticulum; or in other embodiments, Golgi apparatus; or, in other embodiments, lysosomes; or, in other embodiments; peroxisomes, or in, other embodiments, endosomes. In still other embodiments, the vesicular component comprises mitochondria; or in other embodiments, endoplasmic reticulum; or in other embodiments, Golgi apparatus; or, in other embodiments, lysosomes; or, in other embodiments; peroxisomes, or in, other embodiments, endosomes.

In still other embodiments, the vesicular component is enriched in extracellular vesicles, which may be exosomes; or, in other embodiments, microvesicles; or, in other embodiments, exomeres. In yet other embodiments, the vesicular component consists essentially of extracellular vesicles, which may be exosomes; or, in other embodiments, microvesicles; or, in other embodiments, exomeres. In still other embodiments, the vesicular component consists of extracellular vesicles, which may be exosomes, or, in other embodiments, microvesicles. In still other embodiments, the vesicular component comprises extracellular vesicles, which may be exosomes; or, in other embodiments, microvesicles; or, in other embodiments; or, in other embodiments, exomeres; or, in other embodiments, ectosomes.

Microvesicles (also referred to herein as microparticles) are, in various embodiments, identified based on any combination of size (e.g., between 100 nm-1 μm), surface markers, and/or the exposure of the negatively charged phosphatidylserine in the outer membrane (Johnstone et al. and Pan et al.). Methods for isolating microvesicles are known in the art and are described, for example, in Hugel et al. and Zhong W Q et al. and the references cited therein.

In certain embodiments, the described methods comprise isolation of microparticles by centrifugation and optional flow cytometry, for example as described in Burger D et al. or the references cited therein. One such protocol, provided solely for purposes of exemplification, involves a low-speed centrifugation to remove large cellular debris, fluorescent labeling of surface proteins, and cytometry-based sorting. The low-speed centrifugation can be e.g., for 15 minutes at 1500×g. In certain embodiments, the supernatant from this centrifugation is pelleted again, to ensure removal of large debris. Microparticles can then be pelleted, e.g., by centrifugation at 20,000×g for 20 minutes. The pellet is then resuspended and then, to obtain a high-purity preparation, may be stained with a microparticle surface marker (e.g., Annexin) and subjected to flow cytometry. An upper size limit (e.g., 1 micron) may be established using the forward scatter and side scatter parameters, as will be understood by those skilled in the art of flow cytometry.

In other embodiments, the methods comprise isolation of microparticles by centrifugation, for example as described in Braga-Lagache et al., or the references cited therein. One such protocol, provided solely for purposes of exemplification, involves a pre-clearing centrifugation step for 2 min at 16,000×g at RT. The supernatant is then centrifuged at 16000×g and RT for 20-40 min. The supernatant is then aspirated, and the pellets are reconstituted in buffered solution. MP are optionally pelleted again by centrifugation at 16,000×g and RT for 20 min, followed by 1-2 more optional washing steps.

In yet other embodiments, the placental ASC have been treated to increase production of microparticles. Non-limiting examples of such treatments are cultured under conditions of hypoxia, or in other embodiments of serum starvation, for example as described in WO 2015/015490 to Anat Aharon et al, which is hereby incorporated by reference.

Exomeres, in certain embodiments, refers to nonmembranous secreted nanoparticles, which average about 35 nm (nanometers) in size. In other embodiments, the exomeres are secreted nanoparticles that have a size smaller than 50 nm (e.g., 1-50 nm) and a stiffness in the range of 140-820 megapascals (Mpa). Exomeres are described in Zhang H et al.

Exosomes are, in various embodiments, identified based on their size, e.g., 40-100 nm, and/or their particular cup shape (Nilsson J et al.). Methods for isolating exosomes are known in the art and are described, for example, in Conde-Vancells et al. and Koga et al. In some embodiments, exosomes are identified by glycoaffinity capture (Palmissano et al). In other embodiments, exosomes are isolated by immuno isolation, for example as described in Clayton A et al., 2001; Mathias R A et al., 2009; and Crescitelli R et al., 2013.

In certain embodiments, the described methods comprise isolation of exosomes, for example as described in Conde-Vancells et al. and Koga et al., or the references cited therein. One such protocol, provided solely for purposes of exemplification, involved centrifuging samples for 30 min at 1500×g to remove large cellular debris. The resultant supernatants are subjected to filtration on 0.22 μm pore filters, followed by ultra-centrifugation at 10 000×g and 100 000×g for 30 and 60 min, respectively. The resulting pellets are suspended in PBS, pooled, and again ultracentrifuged at 100 000×g for 60 min. The final pellet (containing vesicles) is suspended in 150 μL of PBS, aliquoted and stored at −80° C. For higher-purity preparations, exosomes can be further purified on sucrose-containing gradients (e.g., a 30% sucrose cushion), e.g., as described in Thery C et al. Vesicle preparations are diluted in PBS and under-layered on top of a density cushion composed of pH-buffered 30% sucrose (optionally containing deuterium oxide (D₂O)), around pH 7.4, forming a visible interphase. The samples are ultracentrifuged at 100 000×g at 4° C. for 75 min in a swinging bucket rotor, and the gradient is withdrawn in aliquots from the bottom. Vesicles contained in the 30% sucrose/D₂O cushion are collected, diluted in buffered solution, and optionally centrifuged at 100 000×g to concentrate the contents. Kits for exosome isolation are available commercially, non-limiting examples of which are ExoQuick® reagents, ExoMAX Opti enhancer, and ExoFLOW products, obtainable from System Biosciences (Palo Alto, CA).

In other embodiments, the fraction is enriched in ectosomes, Ectosomes are known in the art, and are described, for example, in Meldolesi J.

In still other embodiments, the subcellular fraction is enriched in micro-RNA (miRNA). In yet other embodiments, the subcellular fraction comprises miRNA. In other embodiments, the subcellular fraction consists essentially of miRNA. In other embodiments, the subcellular fraction consists of miRNA. In some embodiments, the miRNA is encapsulated within a vesicular compartment, while in other embodiments, the miRNA is not encapsulated within a vesicular compartment.

In other embodiments, there is provided a method of enhancing a deficient metabolic function in a subject, comprising the step of administering to the subject a pharmaceutical composition comprising a subcellular fraction of placental ASC, thereby enhancing a deficient metabolic function. In certain embodiments, the subcellular fraction is from cultured placental ASC. In more specific embodiments, the placental ASC are from the same species as the subject. In other embodiments, the placental ASC are xenogenic to the subject.

In other embodiments, there is provided a method of enhancing aerobic respiration in a subject, comprising the step of administering to the subject a pharmaceutical composition comprising mitochondria of placental ASC, thereby enhancing aerobic respiration. In certain embodiments, the placental ASC are cultured placental ASC. In more specific embodiments, the placental ASC are from the same species as the subject. In other embodiments, the placental ASC are xenogenic to the subject.

In still other embodiments, there are provided methods of restoring a deficient metabolic function(s) to a subject in need thereof, comprising the step of administering the described compositions.

In still other embodiments, there are provided methods of restoring mitochondrial function to a subject in need thereof, comprising the step of administering the described compositions.

In certain embodiments, there is provided a method of treating a muscle wasting disorder in a subject in need thereof, the method comprising the step of administering to the subject a pharmaceutical composition comprising a subcellular fraction (e.g., mitochondria, or in other embodiments exosomes) of placental ASC, thereby treating a muscle wasting disorder in the subject. In other embodiments is provided a method of reducing an incidence of a muscle wasting disorder in a subject in need thereof, the method comprising the step of administering to the subject a composition described herein. In still other embodiments is provided a method of reversing the progress of a muscle wasting disorder in a subject in need thereof, the method comprising the step of administering to the subject a composition described herein.

In various embodiments, the pharmaceutical compositions described herein may be administered via intraosseous infusion, intramuscularly, intravenously, subcutaneously, intraperitoneally, intracerebrally, by intracerebroventricular administration, intratracheal administration, intrathecally, or intranasally, each of which represents a separate embodiment.

In certain embodiments, the composition may be administered dermally. One such embodiment is a cream. In more specific embodiments, the cream contains excipients enabling targeting of skin cells, non-limiting examples of which are cells in the basal epidermis. Alternatively or in addition, the cream is indicated for mitochondrial augmentation of skin cells, amelioration of skin aging, smoothing of wrinkles, and cosmetic improvement of pigmentation abnormalities. In some embodiments, the cream comprises a therapeutically effective amount of mitochondria of placental ASC. In still other embodiments, the composition is an ointment.

In various embodiments, the described mitochondrial preparations may further comprise endoplasmic reticulum (ER), and in some embodiments, may also comprise, nuclei, lysosomes, endosomes, or peroxisomes, each of which represents a separate embodiment. In certain embodiments, the mitochondria preparation contains over 40%, over 50%, over 60%, over 70%, over 80%, over 90%, or over 95% mitochondrial protein, as a weight percentage of total protein therein.

In further embodiments, the mitochondria utilized in the described methods and compositions are naked. In other embodiments, the mitochondria are encapsulated. Non-limiting examples of encapsulated mitochondria include those surrounded by a membrane bilayer (Nakajima A et al.) and in extracellular particles (Hayakawa K et al., Phinney et al., 2015).

In other embodiments, there is provided a method of enhancing a compromised metabolic function in a subject in need thereof, comprising contacting the subject with a cell population enriched in a cellular activity, wherein the enriched cell population has been incubated with a composition comprising a subcellular fraction of a cultured placental ASC, thereby enhancing a compromised metabolic function.

In other embodiments, there is provided a method of enhancing aerobic respiration in a subject in need thereof, comprising contacting the subject with a cell population enriched in mitochondrial activity, wherein the enriched cell population has been incubated with a composition comprising a mitochondrion/a of a cultured placental ASC, thereby enhancing aerobic respiration. Methods of monitoring aerobic respiration are known in the art; non-limiting examples of which include assessment of tissue oxygenation.

In still other embodiments, there is provided a method of enhancing a compromised metabolic function in a subject in need thereof, comprising contacting the subject with an enriched cell, wherein the enriched cell has been incubated with a cultured placental ASC, thereby enhancing a compromised metabolic function in a subject. In some embodiments, the compromised metabolic function is aerobic respiration. Alternatively or in addition, the placental ASC have been subject to a process that increases the mitochondrial content of the placental ASC.

The herein-described enriched cell may be, in various embodiments, a stem cell; a neuron; a muscle cell; airway epithelial cell; liver cell; skin epithelial cell, gastro-intestinal epithelial cell or a cell of the reproductive tract. In more specific embodiments, the enriched cell may be a hematopoietic stem cell; a mesenchymal stem cell, or an induced pluripotent stem cell. Alternatively or in addition, the enriched cell may be allogeneic to the subject; or in other embodiments autologous; or in other embodiments, xenogeneic. In other embodiments, the term stem cells may refer to human adult stem cells that exhibit the ability to self-renew and differentiate into other cell types. The human adult stem cells may be, in more specific embodiments, pluripotent stem cells (cells with the potential to produce virtually any human cell type), multipotent stem cells (e.g., cells that can give rise to osteoblasts, myocytes, chondrocytes and adipocytes), or both. In still other embodiments, neural stem cells are encompassed.

In various embodiments, the enriched cells may be of the same species as the subcellular fraction(s) (e.g., the mitochondria), or in other embodiments, of a different species.

In yet other embodiments, there is provided a method of enriching a recipient cell, comprising contacting the recipient cell with a subcellular fraction of a cultured placental ASC, thereby generating an enriched cell. In other embodiments, the method further comprises the step of subsequently administering the enriched cell to a subject. In other embodiments, the recipient cells were obtained from the treated subject. In other embodiments, the recipient cells are allogeneic to the subject. In more specific embodiments, the recipient cells are autologous to the subject, and the subcellular fraction(s) used to enrich the cells are allogeneic. In still other embodiments, the recipient cell is xenogeneic to the subject.

In yet other embodiments, there is provided a method of providing a mitochondrion to a recipient cell, comprising contacting the recipient cell with a composition comprising a mitochondrion of a cultured placental ASC, thereby providing a mitochondrion to a recipient cell. In certain embodiments, the recipient cell is in need of mitochondrial enhancement, non-limiting examples of which are cells from a subject exhibiting hematopoietic dysfunction, acute lung injury, cachexia, or mtDNA defects. In other embodiments, the recipient cell is autologous to and/or was obtained from the treated subject. In other embodiments, the recipient cell is allogeneic to the subject. In still other embodiments, the recipient cell is xenogeneic to the subject. In certain embodiments, the utilized composition is enriched in mitochondria relative to a whole-cell preparation of placental ASC. In yet other embodiments, the composition consists essentially of mitochondria, naked and/or encapsulated, and vehicle. Those skilled in the art will appreciate that appropriate vehicles may typically comprise buffered aqueous solution and mitochondrial preservatives. Solely for purposes of exemplification, a suitable buffer contains 250 mmol/l (millimoles per liter) sucrose, 2 mmol/l KH₂PO₄, 10 mmol/1 MgCl₂, 20 mmol/l K⁺-HEPES buffer, pH 7.2, 0.5 mmol/l K⁺-EGTA, pH 8.0, 5 mmol/l glutamate, 5 mmol/l malate, 8 mmol/l succinate, and 1 mmol/l ADP.

In yet other embodiments, there is provided a method of providing a mitochondrion to a recipient cell, comprising contacting the recipient cell with a pharmaceutical composition comprising placental ASC, wherein the placental ASC have been subject to a process that increases the mitochondrial content of the placental ASC, thereby generating an augmented placental cell; or in other embodiments a process that induces mitochondrial transfer; or in other embodiments a process that induces mitochondria release. Exemplary embodiments of such processes are described herein. In other embodiments, the enriched cell is indicated for administration to a subject; and/or, the method further comprises the step of subsequently administering the enriched cell to a subject, e.g., a subject in need of mitochondrial enrichment. In certain embodiments, the recipient cell is contacted in vivo (e.g., by administering the augmented placental cells to a subject). In other embodiments, the recipient cell is contacted ex vivo (e.g., by coculturing the recipient cells with the augmented placental cells). Ex-vivo-enriched recipient cells are, in some embodiments, subsequently administered to a subject in need thereof.

In yet other embodiments, there is provided an enriched cell, where the enriched cell has been contacted with a subcellular fraction (e.g., mitochondria or extracellular vesicles) of a cultured placental ASC. In certain embodiments, the contacting is performed ex vivo. In more specific embodiments, the contacting comprises coculturing the placental ASC with the target cell, thereby generating an enriched cell. In other embodiments, the contacting comprises contacting the recipient cell or enriched cell with a composition comprising the subcellular fraction, or in other embodiments, the mitochondria.

In yet other embodiments, there is provided a pharmaceutical composition comprising placental ASC, which have been subject to a process that increases the mitochondrial content of the placental ASC. In yet other embodiments, the placental ASC have been subject to a process that increases mitochondrial transfer. In yet other embodiments, the placental ASC have been subject to a process that increases mitochondrial release. Any of the described formulations of populations of placental ASC populations (which are, in some embodiments, subjected to the described process[es]), cell expansion, formulation of the pharmaceutical composition, administration to subjects may be freely combined with this embodiment of a pharmaceutical composition, and each represents a separate embodiment.

In yet other embodiments, there is provided a preparation, which has been prepared by a process comprising (a) incubating placental ASC in a bioreactor; and (b) obtaining a subcellular fraction from the placental ASC. The subcellular fraction may be any fraction described herein, each of which represents a separate embodiment.

In yet other embodiments, there is provided a mitochondrial preparation, which has been prepared by a process comprising (a) incubating placental ASC in a bioreactor; and (b) obtaining mitochondria from the placental ASC.

In yet other embodiments, there is provided a method of producing a mitochondrial preparation, comprising (a) incubating placental ASC in a bioreactor; and (b) obtaining mitochondria from the placental ASC. In various embodiments, the mitochondria are naked, or are encapsulated. Alternatively or in addition, the placental ASC are treated to increase their mitochondrial content, or, in other embodiments, to stimulate mitochondrial extrusion.

In still other embodiments, there is provided a pharmaceutical composition comprising any of the enriched cells described herein.

In still other embodiments, there is provided a pharmaceutical composition comprising the described preparations, which are, in some embodiments, mitochondrial preparations; and are, in other embodiments, other subcellular fractions.

In certain embodiments, naked mitochondria secreted by the described placental ASC are used in the described methods and compositions.

In other embodiments, mitochondria-containing microvesicles secreted by the described ASC (Phinney D G et al., 2015) are used in the described methods and compositions. Methods of isolating mitochondria-containing microvesicles are known in the art, and include, immuno-isolation, for example, immuno-magnetic isolation, e.g., as described in Macheiner T et al., 2016.

Those skilled in the art will appreciate that methods for isolation of mitochondria are known in the art. Non-limiting examples of such methods are described, for example, in Gostimskaya and Galkin 2010 and Claude 1946. Solely for purposes of exemplification, methods for mitochondrial isolation may include: (i) rupturing of cells by mechanical and/or chemical means; (ii) differential centrifugation at low speed (e.g., 700 g, 10 min) to remove debris and extremely large cellular organelles (SPIN 1); followed by (iii) centrifugation at a higher speed (e.g., 12,000 g, 15 min to isolate mitochondria (SPIN 2). A lower speed for SPIN 2 (e.g., 3,000 g for 15 min. produces a purer preparation. Calcium chelators such as EDTA are preferably avoided.

In certain embodiments, the process of isolating subcellular fractions (e.g., vesicular compartments, which may be e.g., mitochondria, exosomes, etc.) comprises chemical lysis of the placental ASC. Non-limiting embodiments of such methods may utilize kits similar to the ThermoFisher Mitochondria Isolation Kit for Cultured Cells, cat no. 89874.

In other embodiments, the process of isolating subcellular fractions (e.g., vesicular compartments, which may be e.g., mitochondria, exosomes, etc.) comprises physical lysis of the placental ASC. Non-limiting embodiments of such methods may utilize physical disruption methods similar to Dounce homogenization.

In still other embodiments, the process of isolating mitochondria comprises immuno-isolation of the mitochondria.

As provided herein, when placental ASC are incubated with target cells, mitochondrial transfer, or in other embodiments, transfer of endoplasmic reticulum, Golgi, peroxisomes, or endosomes, each of which represents a separate embodiment, occurs via nanotubes (Jackson M V et al., Onfelt B et al., Rustom A et al., Wang Y et al.; and references cited therein), which represents an embodiment of the described methods and compositions.

Methods for increasing the mitochondrial content of cells are known in the art, and include, without limitation, incubation with glucose-free medium. In some embodiments, galactose is included in the medium in place of glucose; while in other embodiments, the medium lacks galactose. Galactose, when present, may be present, in certain embodiments, at a concentration of at 1-20 mM (millimolar); or 1-15, 1-13, 1-12, 1-11, 1-10, 2-20, 2-15, 2-13, 2-12, 2-11, 2-10, 3-20, 3-15, 3-13, 3-12, 3-11, 3-10, 5-20, 5-15, 5-13, 5-12, 5-11, or 5-10 mM. Alternatively or in addition, the growth medium is glucose free and contains pyruvate. In still other embodiments, the growth medium is glucose- and galactose-free and contains pyruvate. In more specific embodiments, pyruvate is present at a concentration of 100 nM (nanomolar)-5 mM; 100 nM-4 mM, 100 nM-3 mM, 100 nM-2 mM, 100 nM-1 mM, 200 nM-5 mM, 200 nM-4 mM, 200 nM-3 mM, 200 nM-2 mM, 200 nM-1.5 mM, 200 nM-1 mM, 300 nM-5 mM, 300 nM-4 mM, 300 nM-3 mM, 300 nM-2 mM, 300 nM-1.5 mM, 300 nM-1 mM, 400 nM-5 mM, 300 nM-4 mM, 300 nM-3 mM, 300 nM-2 mM, 300 nM-1.5 mM, 300 nM-1 mM, 500 nM-5 mM, 500 nM-4 mM, 500 nM-3 mM, 500 nM-2 mM, 500 nM-1.5 mM, 500 nM-1 mM, 600-1500 nM, 700-1400 nM, 800-1300 nM, 900-1200 nM, 900-1100 nM, about 1 mM, or 1 mM. In still other embodiments, the placental ASC are incubated at lower oxygen levels than standard culture conditions, e.g., partial pressure of O₂ of 3-10%, 3-9%, 3-8%, 3-7%, 3-6%, 4-10%, 4-9%, 4-8%, 4-7%, 4-6%, 5-6%, or 4-5%. In certain embodiments, low oxygen conditions are combined with glucose-free conditions, each of which is considered a separate embodiment. Other, non-limiting examples of such protocols are described in Mot A I et al., Diers A R et al., and the references cited therein. In other embodiments, the placental ASC are incubated with low doses of a cytotoxic necrotising factor, a non-limiting example of which is the E. coli protein toxin cytotoxic necrotising factor 1 (CNF1), for example as described in Travaglione S et al. and the references cited therein. In other embodiments, the placental ASC are incubated with one or more polyphenols (non-limiting examples of which are resveratrol and/or equol), for example as described in Sergio Davinelli et al. and the references cited therein. In still other embodiments, the placental ASC are incubated with creatine, coenzyme Q10 (CoQ10), or analogues thereof (non-limiting examples of which are idebenone and mitoquinone), for example as described in Zhang Z W et al. and the references cited therein. In other embodiments, the placental ASC are incubated with CHBP (thioether-cyclized helix B peptide; FIG. 6), for example as described in Travaglione S et al. and Wang S et al., and the references cited therein.

In more specific embodiments, the protocol to increase mitochondrial content (“augmentation protocol”) is performed in a bioreactor and/or on a 3D substrate, which may be any of the embodiments of bioreactors and substrates described herein. Alternatively, the augmentation protocol is performed during growth on a 2D substrate; or, in yet other embodiments, both on a 2D substrate and subsequently on a 3D substrate in a bioreactor. In still other embodiments, any of the aforementioned augmentation protocols is performed for 8 hours (h)-10 days (d), 8 h-8 d, 8 h-6 d, 8 h-5 d, 8 h-4 d, 8 h-3 d, 8 h-2 d, 8-36 h, 8-24 h, 8-16 h, 12 h-10 d, 12 h-8 d, 12 h-6 d, 12 h-10 d, 12 h-8 d, 12 h-6 d, 12 h-5 d, 12 h-4 d, 12 h-3 d, 12 h-2 d, 12-36 h, 12-24 h, 18 h-10 d, 18 h-8 d, 18 h-6 d, 18 h-5 d, 18 h-4 d, 18 h-3 d, 18 h-2 d, 18-36 h, 18-24 h, 24 h-10 d, 24 h-8 d, 24 h-6 d, 24 h-5 d, 24 h-4 d, 24 h-3 d, 24 h-2 d, 24-36 h, 36-72 h, 36-60 h, 36-48 h, 48-96 h, 48-80 h, 48-72 h, 2-10 d, 3-10 d, 4-10 d, 5-10 d, 6-10 d, 8-10 d, 2-8 d, 3-8 d, 4-8 d, 5-8 d, 6-8 d, 2-6 d, 3-6 d, 4-6 d, or 5-6 d.

In other embodiments, the placental ASC are cultured in the presence of extracts, or in other embodiments CM, from ischemic cells. Non-limiting example of such protocols are described in Cha et al.

In certain embodiments, the placental ASC, or in other embodiments the target cells, or in other embodiments both cell types, are treated ex vivo with an agent that stimulates mitochondrial transfer (e.g., in the case of co-incubation) or mitochondrial release (e.g., in the case of isolating of mitochondria from placental ASC). Those skilled in the art will appreciate that agents that stimulate mitochondrial transfer are known in the art. Non-limiting examples of such agents include treatment of the recipient cells with Adriamycin/doxorubicin (Yasuda K et al.) or with ethidium bromide or rhodamine 6G treatment to induce temporary or lasting mitochondrial dysfunction (Cho Y M et al.); treatment of mitochondrial donor cells with hydrogen peroxide, serum deprivation, or agents that stimulate F-actin polymerization (Wang Y et al.) or acidified and/or hyperglycemic and/or low-serum medium (a non-limiting example of which is pH 6.6, 50 mM glucose, 2.5% fetal calf serum); treatment with cytokines that stimulate epithelial-to-mesenchymal transition (EMT) (non-limiting embodiments of which are insulin; cholera toxin; hydrocortisone, EGF, FGF, and TGF-β3) (e.g. as described in Emil Lou et al.); and overexpression of Miro-1 in donor cells (Ahmad T et al.). In other embodiments, the placental ASC are treated with TGF-β and IFN-g, e.g., as described in An J H et al. In more specific embodiments, the treatment is non-toxic and/or does not produce any lasting cell damage, non-limiting examples of which are rhodamine 6G treatment, as will be appreciated by those skilled in the art. In still other embodiments, the treatment that stimulates mitochondrial transfer, or in other embodiments mitochondrial release, is performed for 8 h-10 d, 8 h-8 d, 8 h-6 d, 8 h-5 d, 8 h-4 d, 8 h-3 d, 8 h-2 d, 8-36 h, 8-24 h, 8-16 h, 12 h-10 d, 12 h-8 d, 12 h-6 d, 12 h-10 d, 12 h-8 d, 12 h-6 d, 12 h-5 d, 12 h-4 d, 12 h-3 d, 12 h-2 d, 12-36 h, 12-24 h, 18 h-10 d, 18 h-8 d, 18 h-6 d, 18 h-5 d, 18 h-4 d, 18 h-3 d, 18 h-2 d, 18-36 h, 18-24 h, 24 h-10 d, 24 h-8 d, 24 h-6 d, 24 h-5 d, 24 h-4 d, 24 h-3 d, 24 h-2 d, 24-36 h, 36-72 h, 36-60 h, 36-48 h, 48-96 h, 48-80 h, 48-72 h, 2-10 d, 3-10 d, 4-10 d, 5-10 d, 6-10 d, 8-10 d, 2-8 d, 3-8 d, 4-8 d, 5-8 d, 6-8 d, 2-6 d, 3-6 d, 4-6 d, or 5-6 d.

Any of the described formulations of populations of placental ASC populations, methods of cell expansion, formulations of pharmaceutical compositions, and methods of administration to cells or subjects may be freely combined with any of the described processes for increasing mitochondrial content; inducing mitochondrial transfer; or inducing mitochondrial release. Each combination represents a separate embodiment.

In still other embodiments, donor cells are treated with agents that stimulate generation of plasma membrane vesicles (PMVs) via mechanical extrusion (Xu L Q et al.) In yet other embodiments, centrifugation of recipient cells with mitochondria and/or incubation of recipient cells with mitochondria at 37 degrees Celsius is used to enhance mitochondrial uptake (Caicedo A et al., 2015).

In yet other embodiments, a Nanoblade is used to facilitate uptake of mitochondria by recipient cells (Wu T H et al.). In still other embodiments, microinjection is used (Levy S E, et al.), magnetic beads are used, or Pep-1 is used (Chang J C et al., 2013) Each of the described treatments to increase mitochondrial content or facilitate mitochondria export, transfer, or uptake may be performed, in various embodiments, during incubation on a 2D substrate, a 3D substrate, or a combination thereof, and may be freely combined with the described embodiments for cell expansion.

Those skilled in the art will appreciate that methods for administration of subcellular fraction-containing compositions to a subject, and to cells ex vivo, are known in the art, and are described, for example, in Xu L Q et al., Masuzawa A et al., Cowan D B et al., and McCully J D et al., 2009. Methods for monitoring mitochondrial uptake by recipient cells are also known, and include, but are not limited to, Mitoception (Caicedo A et al., 2015), as well as methods for measuring the mitochondrial activity of mitochondrial-transformed cells (Kesner E E et al. and Pacak C A et al.).

Subjects treated by the described methods and compositions include, in various embodiments, subjects suffering from ischemia/reperfusion injury; stroke; mtDNA defects, non-limiting examples of which are LHON (Leber's hereditary optic neuropathy), MELAS (mitochondrial myopathy, encephalomyopathy, lactic acidosis, stroke-like symptoms), Pearson syndrome, Leigh syndrome, NARP (neuropathy, ataxia, retinitis pigmentosa, and ptosis), MERRF (myoclonic epilepsy with ragged red fibers), KSS (Kearns-Sayre Syndrome), MNGIE (myoneurogenic gastrointestinal encephalopathy), Friedreich Ataxia, and Alpers' disease; acute lung injury, ARDS (Lin K C et al.); neuronal dysfunctions (e.g., Parkinson's Disease, ALS (Park J H et al.); muscle wasting, including in some embodiments dysfunctions in skeletal and cardiac muscle in aging patients, and hematopoietic dysfunction. In other embodiments, the treated subject suffers graft-versus-host disease (GvHD). In still other embodiments, the treated subject suffers from another inflammatory or autoimmune disease, non-limiting examples of which are osteoarthritis, Multiple Sclerosis (MS), systemic lupus erythematosus (SLE), rheumatoid arthritis, systemic sclerosis, Sjorgen's syndrome, Myasthenia Gravis (MG), Guillain-Barre Syndrome (GBS), Hashimoto's Thyroiditis (HT), Graves's Disease, Insulin-dependent/Type I Diabetes Mellitus (IDDM), and Inflammatory Bowel Disease. In certain embodiments, the aforementioned disorders are treated with compositions comprising mitochondria from placental ASC, or in other embodiments with cells enriched by mitochondria from placental ASC.

In certain embodiments, lung-targeted formulations utilize aerosolized formulations. Alternatively or in addition, subjects suffering from allergically inflamed lung or asthma are treated with the described methods and compositions. In other embodiments, lung-targeted formulations comprise mitochondria from placental ASC.

In various embodiments, the described compositions are administered to the subject within 1 hour, within 2 hours, within 3 hours, within 4 hours, within 6 hours, within 8 hours, within 10 hours, within 12 hours, within 15 hours, within 18 hours, within 24 hours, within 30 hours, within 36 hours, within 48 hours, within 3 days, within 4 days, within 5 days, within 6 days, within 8 days, within 10 days, within 12 days, or within 20 days of a precipitating event; e.g., acute lung injury or an ischemic event. In other embodiments, the described compositions are administered to alleviate a condition selected from long-term lung damage, ischemia, hematopoietic dysfunction, acute lung injury, cachexia, and mtDNA defects. In more specific embodiments, the described compositions are administered 1-24, 2-24, 3-24, 4-24, 5-24, 6-24, 8-24, 10-24, 12-48, 1-48, 2-48, 3-48, 4-48, 5-48, 6-48, 8-48, 10-48, 12-48, 18-48, 24-48, 1-72, 2-72, 3-72, 4-72, 5-72, 6-72, 8-72, 10-72, 12-72, 18-72, 24-72, or 36-72 hours after the precipitating event (or after the estimated time of the precipitating event, if the exact time is not known). In still other embodiments, the described compositions are administered 3-48, 4-48, 5-48, or 6-48 hours after the precipitating event (or after the estimated time of the precipitating event, if the exact time is not known), to alleviate lung damage, ischemia/reperfusion injury, hematopoietic dysfunction, acute lung injury, cachexia, or a mtDNA defect or disorder.

### ASC and Sources Thereof

In some embodiments, the described ASC (from which, in some embodiments, subcellular fractions are isolated) are placenta-derived. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [non-limiting embodiments of which are phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis.

Placental cells may be obtained, in various embodiments, from a full-term or pre-term placenta. In some embodiments, the placental tissue is optionally minced, followed by enzymatic digestion. Optionally, residual blood is removed from the placenta before cell harvest. This may be done by a variety of methods known to those skilled in the art, for example by perfusion. The term “perfuse” or “perfusion” as used herein refers to the act of pouring or passaging a fluid over or through an organ or tissue. In certain embodiments, the placental tissue may be from any mammal, while in other embodiments, the placental tissue is human. A convenient source of placental tissue is a post-partum placenta (e.g., less than 10 hours after birth); however, a variety of sources of placental tissue or cells may be contemplated by the skilled person. In other embodiments, the placenta is used within 8 h, within 6 h, within 5 h, within 4 h, within 3 h, within 2 h, or within 1 hour of birth. In certain embodiments, the placenta is kept chilled prior to harvest of the cells. In other embodiments, prepartum placental tissue is used. Such tissue may be obtained, for example, from a chorionic villus sampling or by other methods known in the art. Once placental cells are obtained, they are, in certain embodiments, allowed to adhere to the surface of an adherent material to thereby isolate adherent cells. In some embodiments, the donor is 35 years old or younger, while in other embodiments, the donor may be any woman of childbearing age.

Those skilled in the art will appreciate in light of the present disclosure that cells may be, in some embodiments, extracted from a placenta, for example using physical and/or enzymatic tissue disruption, followed by marker-based cell sorting, and then may be subjected to the culturing methods described herein.

Treatment of Cells with Pro-Inflammatory Cytokines

In certain embodiments of the described methods and compositions, the composition of the medium is not varied during the course of the culturing process used to expand the placental ASC from which a subcellular fraction (e.g., mitochondria, extracellular vesicles, etc.) is isolated or, in other embodiments, the source of the placental ASC used per se in the described methods and compositions. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the 3D culturing method used to prepare the ASC comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g., passaging) the cells to a different incubator, before adding the cytokine-containing medium. Those skilled in the art will appreciate, in light of the present disclosure, that the ASC to be used in the described methods may be extracted, in various embodiments, from the placenta, from adipose tissue, or from other sources, as described further herein.

Other embodiments of pro-inflammatory cytokines, and methods comprising same, are described in WO 2017/141181 to Pluristem Ltd, by Zami Aberman et al., which is incorporated by reference herein. In certain embodiments, a subcellular fraction (e.g., mitochondria or extracellular vesicles) of cells expanded in the presence of inflammatory cytokines is used in the described methods and compositions.

### Other Types of Placental ASC

In still other embodiments, described placental ASC (the cells from which a subcellular fraction (e.g., mitochondria, extracellular vesicles, etc.) is isolated or, in other embodiments, the source of the placental ASC used per se in the described methods and compositions) are a placental cell population that is a mixture of fetal-derived placental ASC (also referred to herein as “fetal ASC” or “fetal cells”) and maternal-derived placental ASC (also referred to herein as “maternal ASC” or “maternal cells”), where a majority of the cells are maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g., a Y chromosome in the case of a male fetus).

In other embodiments, the described placental ASC are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the placental cell population is a mixture of fetal and maternal cells, where a majority of the cells are fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g. Falcon, Becton, Dickinson, San Jose, CA) with washing medium. In some embodiments, the tissue treatment includes use of a DNAse, a non-limiting example of which is Benzonase from Merck.

In certain embodiments, the described placental ASC are distinguishable from mesenchymal stromal cells (MSC), which may, in some embodiments, be isolated from bone marrow. In further embodiments, the cells are human MSC as defined by The Mesenchymal and Tissue Stem Cell Committee of the International Society for Cellular Therapy (Dominici et al., 2006), based on the following 3 criteria: 1. Plastic-adherence when maintained in standard culture conditions (a minimal essential medium plus 20% fetal bovine serum (FBS)). 2. Expression of the surface molecules CD105, CD73 and CD90, and lack of expression of CD45, CD34, CD14 or CD11b, CD79α or CD19 and HLA-DR. 3. Ability to differentiate into osteoblasts, adipocytes and chondroblasts in vitro. By contrast, the described placental cells are, in certain embodiments, characterized by a reduced differentiation potential, as exemplified and described further herein.

### Placental ASC Populations Expanded Under Serum-Free and Low-Serum Conditions

In other embodiments, the cell populations used as the source of the described subcellular fraction (e.g. mitochondria or extracellular vesicles) are produced by expanding a population of placental ASC in a medium that contains less than 5% animal serum. In certain embodiments, the cell population contains at least predominantly fetal cells (referred to as a “fetal cell population”), or, in other embodiments, contains at least predominantly maternal cells (a “maternal cell population”). In certain embodiments, the aforementioned medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from any species, provided that the serum stimulates expansion of the ASC population, for example human serum, bovine serum (e.g., fetal bovine serum and calf bovine serum), equine serum, goat serum, and porcine serum.

In other embodiments, the described cell populations are produced by a process comprising: a. incubating the ASC population in a first medium, wherein the 1st medium contains less than 5% animal serum, thereby obtaining a 1st expanded cell population; and b. incubating the 1st expanded cell population in a 2nd medium, wherein the 2nd medium also contains less than 5% animal serum, and wherein one or more activating components are added to the 2nd medium. This 2nd medium can also be referred to herein as an activating medium. In other embodiments, the 1st medium or the 2nd medium, or in other embodiments both the 1st and 2nd medium, is/are serum free. In still other embodiments, the 1st medium contains a 1st basal medium, with the addition of one or more growth factors, collective referred to as the “1st expansion medium” (to which a small concentration of animal serum is optionally added); and the activating medium contains a 2nd basal medium with the addition of one or more growth factors (the “2nd expansion medium”), to which activating component(s) are added. In more specific embodiments, the 2nd expansion medium is identical to the 1st expansion medium; while in other embodiments, the 2nd expansion medium differs from the 1st expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In still other embodiments, ASC are extracted from placenta into serum-containing medium. A non-limiting extraction protocol is described in Example 1 of International Patent Application WO 2016/098061, in the name of Esther Lukasiewicz Hagai et al., published on Jun. 23, 2016, which is incorporated herein by reference in its entirety. Following initial extractions, cells are, in further embodiments, expanded in SRM. For such embodiments, the nomenclature of the aforementioned steps is retained, with the first medium (serum-replacement medium or SRM) called the “first medium”, and the activating medium called the “second [or activating] medium”.

In certain embodiments, the described serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al. In still other embodiments, a chemically-defined medium is utilized.

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

Other SFM and SRM embodiments are disclosed in international patent application publ. no. WO 2019/186471, filed on Mar. 28, 2019, in the name of Lior Raviv et al., which is incorporated herein by reference.

### Other Culture Embodiments

In other embodiments, the placental ASC are cultured in the presence of extracts, or in other embodiments CM, from ischemic cells. Non-limiting example of such protocols are described in Cha et al.

In other embodiments, the placental ASC are subjected to preconditioning or other treatments (e.g., as described herein) before isolation of mitochondria and/or other subcellular fractions from them—after which the mitochondria or subcellular fractions can be used for any of the uses described herein.

In yet other embodiments, the placental ASC are cultured, or in other embodiments incubated, under hypoxic conditions. Methods for hypoxia preconditioning are known in the art; e.g., as described in Bader A M, et al. and the references cited therein; other non-limiting examples of such methods include treatment with 0.1-0.3% O₂, treatment with 0.5% O₂, e.g. for 24 hours; treatment with a 1% O₂ and 5% CO₂ atmosphere, in some embodiments in glucose-free medium, e.g. for 24 hours; culturing at 2%, 3% or 5% 02 for 1-7 days; culturing at 5% O₂ for 2 days; and culturing in 95% O₂. Also encompassed, in other embodiments, are regimens of hypoxia preconditioning (which may be 1-5%, e.g. about 2.5% O₂), reoxygenation (e.g., at ambient conditions, which may be 15-25%, e.g. about 21% O₂), and further hypoxia preconditioning (which may be 1-5%, e.g. about 2.5% O₂); e.g., as described in Hu C and Li L, Liu J et al. 2015, Boyette L B et al., Barros S et al., and the references cited therein.

In yet other embodiments, the placental ASC are subjected to pharmacological preconditioning, non-limiting examples of which are treatment with Deferoxamine (DFO), polyribocytidylic acid, and other toll-like receptor-3 (TLR3) agonists. Protocols for pharmacological preconditioning are known in the art; non-limiting examples are described in Bao Z et al., Liu X et al., and Hu C and Li L; and the references cited therein.

In yet other embodiments, the placental ASC are subjected to preconditioning with one or more hormones, non-limiting examples of which are oxytocin, melatonin, all-trans retinoic acid, SDF-1/CXCR4 (Uniprot Accession No. P61073), Oncostatin M (Uniprot Accession No. P13725), and TGF-beta-1 (Uniprot Accession No. P01137), interferon-gamma (Uniprot Accession No. P01579), and migration inhibitory factor (Uniprot Accession No. P14174). Protocols for hormone preconditioning are known in the art; non-limiting examples of such methods are described in Tang Y et al., Pourjafar M et al., Lan Y W et al., Li D et al., Duijvestein M et al., Xia W et al., Hu C and Li L; and the references cited therein.

In still other embodiments, the placental ASC are subjected to preconditioning with laser light, pulsed electromagnetic fields (PEMF), or nanoparticles and/or microparticles (e.g., silica particles). Protocols for such treatments are known in the art, and non-limiting examples are described in Miranda J M et al., Ross C L et al., Kim K J et al., and the references cited therein.

In yet other embodiments, there is provided a method of preconditioning placental ASC, comprising treating the placental ASC as described herein. In certain embodiments, the conditioned ASC are indicated for treating respiratory distress syndrome. In other embodiments, the ASC are indicated for other therapeutic uses, for example ischemia, or in other embodiments, an ischemic disorder, non-limiting examples of which are peripheral arterial disease (PAD), critical limb ischemia (CLI), lower extremity ischemia, stroke, ischemic vascular disease, vascular disease of the kidney, ischemic heart disease, myocardial ischemia, coronary artery disease (CAD), atherosclerotic cardiovascular disease, Buerger's disease, and ischemic renal disease; autoimmune diseases, non-limiting examples of which are graft-versus-host disease (GvHD), osteoarthritis, Multiple Sclerosis (MS), systemic lupus erythematosus (SLE), rheumatoid arthritis, systemic sclerosis, Sjorgen's syndrome, Myasthenia Gravis (MG), Guillain-Barre Syndrome (GBS), Hashimoto's Thyroiditis (HT), Graves's Disease, Insulin-dependent [Type I] Diabetes Mellitus (IDDM), and Inflammatory Bowel Disease (IBD); preeclampsia; support of hematopoietic stem cell (HSC) transplantation; support of recovery from myeloablative treatments, non-limiting examples of which are radiation and chemotherapy; other hematopoietic indications, non-limiting examples of which are delayed, failed, or incomplete engraftment of an HSC transplant, myelodysplastic syndrome, acute myeloid leukemia (AML), oligoblastic leukemia, GI toxicity in a subject with AML, and aplastic anemia; connective tissue damage; tendon injury; psoriasis: neuropathic pain, e.g., resulting from peripheral nerve injury; and neurodegenerative diseases, e.g., Alzheimer's Disease, Parkinson's, Huntington's, and Amyotrophic Lateral Sclerosis (ALS). In other embodiments, the described preconditioning methods may be freely combined with other culturing and cell expansion methods described herein. In certain embodiments, the placental cells used for the described hematopoietic indications are fetal cells.

In yet other embodiments, there is provided a preparation comprising exosomes derived from placental ASC, for treating ischemia, or in other embodiments, an ischemic disorder. In certain embodiments, the ischemic disorder is selected from PAD, CLI, lower extremity ischemia, stroke, ischemic vascular disease, vascular disease of the kidney, ischemic heart disease, myocardial ischemia, CAD, atherosclerotic cardiovascular disease, Buerger's disease, and ischemic renal disease.

In yet other embodiments, there is provided a preparation comprising exosomes derived from placental ASC, for treating an auto-immune disorder, or in other embodiments, an inflammatory disorder. In certain embodiments, the auto-immune disorder is selected from GvHD, osteoarthritis, MS, SLE, rheumatoid arthritis, systemic sclerosis, Sjorgen's syndrome, MS, MG, GBS, HT, Graves's Disease, (Type I) IDDM, and IBD.

### Surface Markers and Additional Characteristics of ASC

Alternatively or additionally, the described ASC (which are used as the source of subcellular fractions) may express a marker or a collection of markers (e.g., a surface marker) characteristic of MSC or mesenchymal-like stromal cells. In some embodiments, the ASC express some or all of the following markers: CD105 (UniProtKB Accession No. P17813), CD29 (Accession No. P05556), CD44 (Accession No. P16070), CD73 (Accession No. P21589), and CD90 (Accession No. P04216). In some embodiments, the ASC do not express some or all of the following markers: CD3 (e.g. Accession Nos. P09693 [gamma chain]P04234 [delta chain], P07766 [epsilon chain], and P20963 [zeta chain]), CD4 (Accession No. P01730), CD11b (Accession No. P11215), CD14 (Accession No. P08571), CD19 (Accession No. P15391), and/or CD34 (Accession No. P28906). In more specific embodiments, the ASC also lack expression of CD5 (Accession No. P06127), CD20 (Accession No. P11836), CD45 (Accession No. P08575), CD79-alpha (Accession No. B5QTD1), CD80 (Accession No. P33681), and/or HLA-DR (e.g. Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g., at least 20%) of a population of cells express a marker of interest, and a significant percentage do not express the marker.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

A cell is said to express a protein or factor if the presence of protein or factor is detectable by standard methods, an example of which is a detectable signal using fluorescence-activated cell sorting (FACS), relative to an isotype control. Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% FBS+2 mM L-Glutamine), added to each well of a 6 well-plate, and cultured for 24 hrs. in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

According to some embodiments, the described ASC are capable of suppressing an immune reaction in the subject. Methods of determining the immunosuppressive capability of a cell population are well known to those skilled in the art, with exemplary methods described in Example 3 of PCT Publication No. WO 2009/144720, which is incorporated herein by reference in its entirety. For example, in an exemplary, non-limiting mixed lymphocyte reaction (MLR) assay, irradiated cord blood (iCB) cells, for example human cells or cells from another species, are incubated with peripheral blood-derived monocytes (PBMC; e.g., human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In still other embodiments, the ASC secrete immunoregulatory factor(s). In certain embodiments, the ASC secrete a factor selected from TNF-beta (UniProt identifier P01374) and Leukemia inhibitory factor (LIF; UniProt identifier P15018). In other embodiments, ASC secrete a factor selected from MCP-1 (CCL2), Osteoprotegerin, MIF (Macrophage migration inhibitory factor; Accession No. P14174), GDF-15, SDF-1 alpha, GROa (Growth-regulated alpha protein; Accession No. P09341), beta2-Microglobulin, IL-6, IL-8 (UniProt identifier P10145), TNF-beta, ENA78/CXCL5, eotaxin/CCL11 (Accession No. P51671), and MCP-3 (CCL7). In certain embodiments, the ASC secrete MCP-1, Osteoprotegerin, MIF, GDF-15, SDF-1 alpha, GROa, beta2-Microglobulin, IL-6, IL-8, TNF-beta, and MCP-3, which were found to be secreted by maternal cells. In other embodiments, the ASC secrete MCP-1, Osteoprotegerin, MIF, GDF-15, SDF-1 alpha, beta2-Microglobulin, IL-6, IL-8, ENA78, eotaxin, and MCP-3, which were found to be secreted by fetal cells. UniProt entries in this paragraph were accessed on Mar. 23, 2017.

In yet other embodiments, the ASC secrete anti-fibrotic factor(s). In certain embodiments, the ASC secrete a factor selected from Serpin E1 (Plasminogen activator inhibitor 1; Uniprot Accession No. P05121) and uPAR (Urokinase plasminogen activator surface receptor; Uniprot Accession No. Q03405). In other embodiments, the ASC secrete factors that facilitate. In still other embodiments, the ASC secrete Serpin E1 and uPAR, which were found to be secreted by maternal and fetal cells. All UniProt entries in this paragraph were accessed on Apr. 3, 2017.

In other embodiments, the ASC secrete a factor(s) that promotes extracellular matrix (ECM) remodeling. In certain embodiments, the ASC secrete a factor selected from TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells.

In general, in certain embodiments, the described ASC exhibit a spindle shape when cultured under 2D conditions, or more specifically, are spindle in shape, with a flat, polygonal morphology, and are 15-19 μM in diameter. Alternatively or in addition, at least 90% of the cells are Oct-4 minus, as assessed by FACS. In certain embodiments, further steps of purification or enrichment for ASC may be performed. Such methods include, but are not limited to, FACS using ASC marker expression. In other embodiments, the described cells have not been subject to any type of cell sorting in the process used to isolate them. Cell sorting, in this context, refers to any a procedure, whether manual, automated, etc., that selects cells based on their expression of one or more markers, their lack of expression of one or more markers, or a combination thereof. Those skilled in the art will appreciate that data from one or more markers can be used individually or in combination in the sorting process.

Alternatively or in addition, the ASC (a) have a Population Doubling Level (PDL) of no more than 25; (b) stimulate endothelial cell proliferation and/or bone marrow migration in in vitro assays (for example, as described herein); (c) secrete, in various embodiments, at least 1, at least 2, at least 3, at least 4, at least 5, at least 6, or all 7 of IL-10, VEGF, Angiogenin, Osteopontin, IL-6, IL-8, MCP-1; (d) exhibit normal karyotype; (e) exhibit expression (in various embodiments, in at least 80%, 85%, 90%, 93%, 95%, 97%, or 98% of the cells) of CD105, CD73, CD29, and CD90; (f) exhibit lack of expression (in various embodiments, in at least 90%, 93%, 95%, 97%, 98%, 99%, 99.5%, 99.7%, or 99.75% of the cells) of CD14, CD19, CD31, CD34, CD45, HLA-DR, and CD235; or any combination of 2 or more of characteristics a-f, each of which represents a separate embodiment. Alternatively or in addition, less than 5%, less than 10%, less than 20%, less than 30%, less than 40%, less than 50%, more than 60%, more than 70%, more than 80%, more than 90%, or more than 95% of the cells express CD200. These possibilities may be independently combined with characteristics a-f and combinations thereof, each of which represents a separate embodiment.

In other embodiments, each of CD29, CD73, CD90, and CD105 is expressed by more than 80% of the ASC in each of the populations; and over 90% (or in other embodiments, over 95%, or over 98%) of the cells in each population are resistant to osteogenesis, as described in WO 2016/098061, which is incorporated herein by reference. In some embodiments, differentiation into osteocytes is assessed by incubation for 17 days with a solution containing 0.1 mcM dexamethasone, 0.2 mM ascorbic acid, and 10 mM glycerol-2-phosphate, in plates coated with vitronectin and collagen (standard osteogenesis induction conditions). In yet other embodiments, each of CD34, CD39, and CD106 is expressed by less than 10% of the cells; less than 20% of the cells highly express CD56; and the cells do not differentiate into osteocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the standard conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (modified osteogenesis induction conditions). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, 40%, 30%, 20%, 10%, or 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates, as provided herein.

In other embodiments, each of CD29, CD73, CD90, and CD105 is expressed by more than 80% of each of the ASC populations; and over 90% (or in other embodiments, over 95%, or over 98%) of the cells in each population are resistant to adipogenesis, as described in WO 2016/098061, which is incorporated herein by reference. In some embodiments, differentiation into adipocytes is assessed by incubation in adipogenesis induction medium, i.e., a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (standard adipogenesis induction conditions). In yet other embodiments, each of CD34, CD39, and CD106 is expressed by less than 10% of the cells; less than 20% of the cells highly express CD56; and the cells do not differentiate into adipocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, under the standard conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (modified adipogenic conditions). In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the modified conditions. The aforementioned solutions will typically contain cell culture medium such as DMEM+10% serum or the like, as will be appreciated by those skilled in the art. In various embodiments, the cell population may be less than 50%, less than 40%, less than 30%, less than 20%, or less than 10%, or less than 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates.

In still other embodiments, the described ASC possess any other marker phenotype, other characteristic (e.g. secretion of factor(s), differentiation capability, resistance to differentiation, inhibition of T-cell proliferation, or stimulation of myoblast proliferation), or combination thereof that is mentioned and/or described in international patent application publ. no. WO 2019/239295, filed Jun. 10, 2019, to Zami Aberman et al, which is incorporated herein by reference.

In still other embodiments, the cells may be allogeneic, or in other embodiments, the cells may be autologous. In other embodiments, the cells may be fresh or, in other embodiments, frozen (for example, cryo-preserved).

In certain embodiments, any of the aforementioned ASC populations are used in the described methods and compositions. In other embodiments, subcellular fractions obtained from the cells are used in the described methods and compositions. Each population may be freely combined with each of the described treatments, and each combination represents a separate embodiment. Furthermore, the cells utilized to generate fractions or contained in the composition can be, in various embodiments, autologous, allogeneic, or xenogeneic to the treated subject. Each type of cell may be freely combined with the therapeutic embodiments mentioned herein.

### Additional Method Characteristics for Preparation of ASC

In certain embodiments, the ASC from which a subcellular fraction (e.g. mitochondria, or extracellular vesicles) are isolated have been subject to a 3D incubation, as described further herein. In more specific embodiments, the ASC have been incubated in a 2D adherent-cell culture apparatus, prior to the step of 3D culturing. In some embodiments, cells, following extracting, in some embodiments, from placenta, from adipose tissue, or from other tissues, are then subjected to incubation in a 2D adherent-cell culture apparatus, followed by the described 3D culturing steps.

The terms “two-dimensional culture” and “2D culture” refer to cell culture conditions compatible with cell growth, which allow the cells to grow in a monolayer. Suitable apparatuses, referred to as “2D culture apparatus[es]”, typically have flat or curved growth surfaces (also referred to as a “two-dimensional substrate[s]” or “2D substrate[s]”), in some embodiments comprising an adherent material. Non-limiting examples of 2D culture apparatuses are cell culture dishes and plates. Included in this definition are multi-layer trays, such as Cell Factory™ manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to cell culture conditions compatible with cell growth, which allow the cells to grow in a 3D orientation relative to one another. Suitable apparatuses, referred to as “three-dimensional [or 3D] culture apparatus[es]”, typically have a 3D growth surface (also referred to as a “three-dimensional substrate” or “3D substrate”), in some embodiments comprising an adherent material, which is present in the 3D culture apparatus, e.g., the bioreactor.

Placenta-derived cells can be propagated, in some embodiments, by using a combination of 2D and 3D culturing conditions. Certain, non-limiting embodiments of conditions for propagating adherent cells in 2D and 3D culture are further described hereinbelow and in the Examples section which follows. Others are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of synthetic adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other non-limiting examples of adherent materials are described in Int. Appl. Publ. No. WO 2019/239295 to Zami Aberman et al., which is hereby incorporated by reference.

In other embodiments, the length of 3D culturing is at least 4 days; between 4-12 days; in other embodiments between 4-11 days; in other embodiments between 4-10 days; in other embodiments between 4-9 days; in other embodiments between 5-9 days; in other embodiments between 5-8 days; in other embodiments between 6-8 days; or in other embodiments between 5-7 days. In other embodiments, the 3D culturing is performed for 5-15 cell doublings, in other embodiments 5-14, 5-13, 5-12, 5-11, 5-10, 6-15, 6-14, 6-13, 6-12, 6-11, or 6-10 doublings.

### Systems for Cell Expansion

In certain embodiments, the subcellular fraction (e.g., mitochondria or extracellular vesicles) used in the described methods and compositions are isolated from ASC expanded on a 2D substrate, non-limiting of examples of which are described herein. In other embodiments, the subcellular fraction(s) are isolated from ASC expanded on a 3D substrate, non-limiting of examples of which are described herein. In other embodiments, the subcellular fraction(s) are isolated from ASC expanded in a bioreactor on a 3D substrate.

In certain embodiments, as mentioned, 3D culturing can be performed in a 3D bioreactor. In some embodiments, the 3D bioreactor comprises a container for holding medium and a 3D attachment substrate disposed therein, and a control apparatus, for controlling pH, temperature, and oxygen levels and optionally other parameters. The terms attachment substrate and growth substrate are interchangeable. In certain embodiments, the attachment substrate is in the form of carriers, which comprise, in more specific embodiments, a surface comprising a synthetic adherent material. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

As provided herein, a 3D bioreactor is capable, in certain embodiments, of 3D expansion of ASC under controlled conditions (e.g., pH, temperature and oxygen levels) and with growth medium perfusion, which in some embodiments is constant perfusion, and in other embodiments is adjusted in order to maintain target levels of glucose or other components. Furthermore, cultures can be directly monitored for concentrations of glucose, lactate, glutamine, glutamate and ammonium. Glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, NJ). Additional, non-limiting examples of bioreactors are described in Int. Appl. Publ. No. WO 2019/239295 to Zami Aberman et al., which is hereby incorporated by reference.

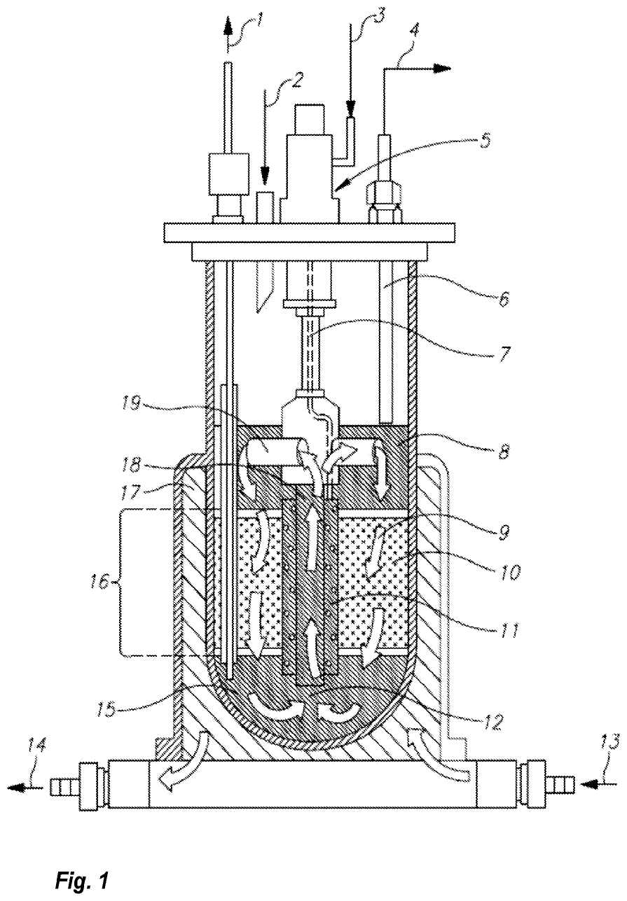

Another exemplary, non-limiting bioreactor, the Celligen 310 Bioreactor, is depicted in FIG. 1. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In some embodiments, temperature, pH, dissolved oxygen concentration, etc., are set prior to inoculation. In further embodiments, a slow initial stirring rate is used to promote cell attachment, then the stirring rate is increased. Alternatively or in addition, perfusion is initiated by adding fresh medium via an external port (2). Optionally, metabolic products are harvested from the cell-free medium above the basket (8). In some embodiments, rotation of the impeller creates negative pressure in the draft-tube (18), which pulls cell-free effluent from a reservoir (15) through the draft tube, then through an impeller port (19), causing medium to circulate (12) uniformly in a continuous loop. In other embodiments, adjustment of a tube (6) controls the liquid level; an external opening (4) of this tube is used in some embodiments for harvesting cells. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or additionally, sparged gas confined to the remote chamber is absorbed by the nutrient medium, which washes over the immobilized cells. In other embodiments, a water jacket (17) is present, with ports for moving the jacket water in (13) and out (14).

In certain embodiments, a perfused bioreactor is used, wherein the perfusion chamber contains carriers.

In some embodiments, the carriers in the perfused bioreactor are packed, for example forming a packed bed, which is submerged in a nutrient medium. Alternatively or in addition, the carriers comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect are described elsewhere herein. In more particular embodiments, the material is selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials are described elsewhere herein. Other embodiments include Matrigel™, an extra-cellular matrix component (e.g., Fibronectin, Chondronectin, Laminin), and a collagen.

In other embodiments, prefabricated or rigid scaffolds are utilized. Such scaffolds require, in some embodiments, migration of cells into the scaffold, after cell seeding. In other embodiments, physically crosslinked scaffolds may be utilized, which are, in further embodiments, gels that are formed via reversible changes in pH or temperature.

In other embodiments, cells are produced using a packed-bed spinner flask. In more specific embodiments, the packed bed may comprise a spinner flask and a magnetic stirrer. The spinner flask may be fitted, in some embodiments, with a packed bed apparatus, which may be, in more specific embodiments, a fibrous matrix; or, in more specific embodiments, a non-woven fibrous matrix. In other embodiments, the fibrous matrix comprises polyester, or comprises at least about 50% polyester. In still other embodiments, the non-woven fibrous matrix comprises polyester, or comprises at least about 50% polyester.

In still other embodiments, the matrix is similar to the Celligen™ Plug Flow bioreactor which is, in certain embodiments, packed with Fibra-Cel® carriers (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, 450-650 mg\liter, 475-625 mg\liter, 500-600 mg\liter, or between 525-575 mg\liter. In yet other embodiments, at the end of the culture process, carriers are removed from the packed bed, washed with isotonic buffer, and cells are removed from the carriers by agitation and/or enzymatic digestion.

In certain embodiments, the bioreactor is seeded at a concentration of between 10,000-2,000,000 cells/ml of medium; or in other embodiments 20,000-2,000,000 cells/ml, 30,000-1,500,000, 40,000-1,400,000, 50,000-1,300,000, 60,000-1,200,000, 70,000-1,100,000, 80,000-1,000,000, 80,000-900,000, 80,000-800,000, 80,000-700,000, 80,000-600,000, 80,000-500,000, 80,000-400,000, 90,000-300,000, 90,000-250,000, 90,000-200,000, 100,000-200,000, 110,000-1,900,000, 120,000-1,800,000, 130,000-1,700,000, or 140,000-1,600,000 cells/ml.

In still other embodiments, between 1-20×10⁶ cells per gram (gr) of carrier (substrate) are seeded; or in other embodiments 1.5-20×10⁶ cells/gr carrier, 1.5-18×10⁶, 1.8-18×10⁶, 2-18×10⁶, 3-18×10⁶, 2.5-15×10⁶, 3-15×10⁶, 3-14×10⁶, 3-12×10⁶, 3.5-12×10⁶, 3-10×10⁶, 3-9×10⁶, 4-9×10⁶, 4-8×10⁶, 4-7×10⁶, or 4.5-6.5×10⁶ cells/gr carrier.

In certain embodiments, cell harvest from the bioreactor is performed when at least about 10%, 12%, 14%, 16%, 18%, 20%, 22%, 24%, 26%, 28%, or 30% of the cells are in the S and G2/M phases (collectively), as can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In other embodiments, over 5×10⁵, over 7×10⁵, over 8×10⁵, over 9×10⁵, over 10⁶, over 1.5×10⁶, over 2×10⁶, over 3×10⁶, over 4×10⁶, or over 5×10⁶ viable cells are removed per milliliter of the growth medium in the bioreactor. In still other embodiments, between 5×10⁵-1.5×10⁷, 7×10⁵-1.5×10⁷, 8×10⁵-1.5×10⁷, 1×10⁶-1.5×10⁷, 5×10⁵-1×10⁷, 7×10⁵-1×10⁷, 8×10⁵-1×10⁷, 1×10⁶-1×10⁷, 1.2×10⁶-1×10⁷, or between 2×10⁶-1×10⁷ viable cells are removed per milliliter of the growth medium in the bioreactor.

In other embodiments, incubation of ASC may comprise microcarriers, which may, in certain embodiments, be inside a bioreactor. Microcarriers are known to those skilled in the art, and are described, for example in U.S. Pat. Nos. 8,828,720, 7,531,334, 5,006,467, which are incorporated herein by reference. Microcarriers are also commercially available, for example as Cytodex™ (available from Pharmacia Fine Chemicals, Inc.), Superbeads (commercially available from Flow Labs, Inc.), and DE-52 and DE-53 (commercially available from Whatman, Inc.). In certain embodiments, the ASC may be incubated in a 2D apparatus, for example tissue culture plates or dishes, prior to incubation in microcarriers. In other embodiments, the ASC are not incubated in a 2D apparatus prior to incubation in microcarriers. In certain embodiments, the microcarriers are packed inside a bioreactor.

In certain embodiments, further steps of purification or enrichment for ASC may be performed. Such methods include, but are not limited to, cell sorting using markers for ASC and/or, in various embodiments, mesenchymal stromal cells or mesenchymal-like ASC.

Cell sorting, in this context, refers to any procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

In certain embodiments, cells are removed from a 3D matrix while the matrix remains within the bioreactor. In certain embodiments, at least about 10%, at least 12%, at least 14%, at least 16%, at least 18%, at least 20%, at least 22%, at least 24%, at least 26%, at least 28%, or at least 30% of the cells are in the S and G2/M phases (collectively), at the time of cell harvest from the bioreactor. Cell cycle phases can be assayed by various methods known in the art, for example FACS detection. Typically, in the case of FACS, the percentage of cells in S and G2/M phase is expressed as the percentage of the live cells, after gating for live cells, for example using a forward scatter/side scatter gate. Those skilled in the art will appreciate that the percentage of cells in these phases correlates with the percentage of proliferating cells. In some cases, allowing the cells to remain in the bioreactor significantly past their logarithmic growth phase causes a reduction in the number of cells that are proliferating.

In general, the described step of harvesting cells, e.g., from a matrix or carriers, may constitute a step prior to isolation of the described subcellular fractions from the cells.

In certain embodiments, the process of harvesting cells from a matrix or carriers comprises vibration or agitation, for example as described in PCT International Application Publ. No. WO 2012/140519, which is incorporated herein by reference. In certain embodiments, to effect harvesting, the cells are agitated at 0.7-6 Hertz, or in other embodiments 1-3 Hertz, during, or in other embodiments during and after, treatment with a protease, optionally also comprising a calcium chelator. In certain embodiments, the carriers containing the cells are agitated at 0.7-6 Hertz, or in other embodiments 1-3 Hertz, while submerged in a solution or medium comprising a protease, optionally also comprising a calcium chelator. Non-limiting examples of a protease plus a calcium chelator are trypsin, or another enzyme with similar activity, optionally in combination with another enzyme, non-limiting examples of which are Collagenase Types I, II, III, and IV, with EDTA. Enzymes with similar activity to trypsin are known in the art; non-limiting examples are TrypLE™, a fungal trypsin-like protease, and Collagenase, Types I, II, III, and IV, which are available commercially from Life Technologies. Enzymes with similar activity to collagenase are known in the art; non-limiting examples are Dispase I and Dispase II, which are available commercially from Sigma-Aldrich. In still other embodiments, the cells are harvested by a process comprising an optional wash step, followed by incubation with collagenase, followed by incubation with trypsin. In various embodiments, at least one, at least two, or all three of the aforementioned steps comprise agitation. In more specific embodiments, the total duration of agitation during and/or after treatment with protease plus a calcium chelator is between 2-10 minutes, in other embodiments between 3-9 minutes, in other embodiments between 3-8 minutes, and in still other embodiments between 3-7 minutes. In still other embodiments, the cells are subjected to agitation at 0.7-6 Hertz, or in other embodiments 1-3 Hertz, during the wash step before the protease and calcium chelator are added. Alternatively or in addition, the ASC are expanded using an adherent material in a container, which is in turn disposed within a bioreactor chamber; and an apparatus is used to impart a reciprocating motion to the container relative to the bioreactor chamber, wherein the apparatus is configured to move the container in a manner causing cells attached to the adherent material to detach from the adherent material. In more specific embodiments, the vibrator comprises one or more controls for adjusting amplitude and frequency of the reciprocating motion. Alternatively or in addition, the adherent material is a 3D substrate, which comprises, in some embodiments, carriers comprising a synthetic adherent material.

### Solutions and Media

Those skilled in the art will appreciate that a variety of isotonic buffers may be used for washing cells and similar uses. Hank's Balanced Salt Solution (HBSS; Life Technologies) is only one of many buffers that may be used.

Non-limiting examples of base media useful in 2D and 3D culturing of ASC include Minimum Essential Medium Eagle, F10(HAM), F12 (HAM), Dulbecco's Modified Eagle Medium (DMEM), and others described in WO 2018/185584, which is incorporated herein by reference. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be fetal bovine serum, or in other embodiments another animal serum. In still other embodiments, the medium is serum-free.

Alternatively or in addition, the medium may be supplemented by growth factors, vitamins (e.g., ascorbic acid), cytokines, salts (e.g., B-glycerophosphate), steroids (e.g., dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, i.e., the 2D growth medium and the 3D growth medium, may be independently selected from each of the described embodiments relating to medium composition. In various embodiments, any medium suitable for growth of cells in a standard tissue apparatus and/or a bioreactor may be used.

In yet other embodiments, the placental ASC are grown as spheroids as part of the preparation process. Examples of conditions that favor spheroid formation (“spheroid conditions”) are known to those skilled in the art. Non-limiting examples of such conditions include incubation in hydrogels (e.g., polyethylene glycol hydrogels), for example as described in Cha et al. and the references cited therein. In more specific embodiments, the cells are cultured in spheroid conditions following 2D culture and prior to bioreactor culture, from which the cells and their subcellular fractions are isolated; or the cells are cultured in 2D culture, followed by spheroid conditions, from which the cells and their subcellular fractions are isolated; or the cells are cultured in spheroid conditions following 2D culture and prior to additional 2D culture, which is then followed by bioreactor culture, from which the cells and their subcellular fractions are isolated; or the cells are cultured in spheroid conditions following 2D culture and prior to additional 2D culture, from which the cells and their subcellular fractions are isolated, each of which represents a separate embodiment. In still other embodiments, the cells are cultured in spheroid conditions following 2D culture and prior to bioreactor culture, from which the cells are isolated and used for ex-vivo incubation, or in other embodiments for direct therapeutic applications; or the cells are cultured in spheroid conditions following 2D culture, from which the cells are isolated and used for ex-vivo incubation, or in other embodiments for direct therapeutic applications; or the cells are cultured in spheroid conditions following 2D culture and prior to additional 2D culture, which is then followed by bioreactor culture, from which the cells are isolated and used for ex-vivo incubation, or in other embodiments for direct therapeutic applications; or the cells are cultured in spheroid conditions following 2D culture and prior to additional 2D culture, from which the cells are isolated and used for ex-vivo incubation, or in other embodiments for direct therapeutic applications, each of which represents a separate embodiment.

It will also be appreciated that in certain embodiments, when the described ASC are intended for administration to a human subject, the cells and the culture medium (e.g., with the above-described medium additives) are substantially xeno-free, i.e., devoid of any animal contaminants. For example, the culture medium can be supplemented with a serum-replacement, human serum and/or synthetic or recombinantly produced factors.

In some embodiments, the subcellular fraction (e.g., mitochondria or extracellular vesicles) is isolated from cells following harvest of the cells from a 3D bioreactor in which the ASC have been incubated. Alternatively or in addition, the cells are cryopreserved, and then are thawed as needed, after which the subcellular fraction(s) are isolated. In other embodiments, the cells are cryopreserved, and then are thawed as needed, after which the ASC are co-cultured with recipient cells. In some embodiments, after 3D culture and thawing, the cells are cultured in 2D culture, from which the subcellular fraction(s) are isolated.

In yet other embodiments, the placental ASC are immortalized, thereby generating a cell line. The cell line is used, in other embodiments, to form an optionally frozen cell bank, which is, in turn, used as a source of subcellular fractions. In other embodiments, the cells from the cell bank are thawed as needed and co-cultured with recipient cells, thereby generating enriched cells. Methods for cell immortalization are known in the art. Non-limiting examples of such methods are described, for example, in the handbook titled General Guidelines for Cell Immortalization, published by Applied Biologic Materials Inc. (Richmond, BC, Canada), and the references cited therein.

In other embodiments, there is provided a cell line of immortalized placental ASC. In still other embodiments, there is provided a subcellular fraction derived from a cell line of immortalized placental ASC. In other embodiments, there is provided a pharmaceutical composition comprising a cell line of immortalized placental ASC. In still other embodiments, there is provided a pharmaceutical composition comprising a subcellular fraction derived from a cell line of immortalized placental ASC. In certain embodiments, the cell line has been subjected to treatment to increase mitochondria content, prior to generation of the subcellular fraction. Alternatively or in addition, any of the aforementioned pharmaceutical compositions may be cryopreserved, optionally in single-dose aliquots. The subcellular fraction may be any subcellular fraction described herein. The placental ASC (prior to immortalization) may be any placental ASC described herein. Each embodiment of a subcellular fraction, of a placental ASC (prior to immortalization), and each combination thereof, represents a separate embodiment.

In other embodiments, the subcellular fractions are cryopreserved (optionally in aliquots), then are thawed as needed for administration to a subject. In still other embodiments, the subcellular fractions themselves are cryopreserved, then are thawed as needed for ex-vivo enrichment of recipient cells. In certain embodiments, such products have the advantage of not requiring deep-freezing to maintain their viability, in contrast to cells, which in some circumstances must be stored at −80° C., −150° C., or even colder temperatures, to maintain long-term viability.

In still other embodiments, the described mitochondria or exosomes of placental ASC comprise mtDNA (mitochondria DNA). In yet other embodiments, mtDNA that is not encapsulated in a vesicular compartment is utilized as a subcellular fraction.

### Pharmaceutical Compositions

The described enriched cells and subcellular fractions (e.g., mitochondria or, in other embodiments, extracellular vesicles), or in other embodiments mitochondria-enriched cellular fractions, can be administered as a part of a pharmaceutical composition, e.g., that further comprises one or more pharmaceutically acceptable carriers. Herein, “pharmaceutically acceptable carrier” refers to a carrier or a diluent. In some embodiments, a pharmaceutically acceptable carrier does not cause significant irritation to a subject. In some embodiments, a pharmaceutically acceptable carrier does not abrogate the biological activity and properties of administered cells. Examples, without limitations, of carriers are aqueous buffers. In some embodiments, the pharmaceutical carrier is an aqueous saline solution.

In other embodiments, compositions are provided herein that comprise enriched cells or subcellular fractions (e.g., mitochondria or, in other embodiments, extracellular vesicles), or in other embodiments mitochondria-enriched cellular fractions, in combination with an excipient, e.g., a pharmacologically acceptable excipient. In further embodiments, the excipient is an osmoprotectant or cryoprotectant, an agent that protects cell or fractions from the damaging effect of freezing and ice formation, which may in some embodiments be a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively or in addition, the composition is frozen. In more specific embodiments, DMSO may be present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g., human serum albumin. The fractions may be any embodiment of subcellular fractions mentioned herein, each of which represents a separate embodiment.

In other embodiments, the described pharmaceutical compositions comprise an aqueous buffer and mitochondrial preservatives, which are known to those skilled in the art.

In other embodiments, there are provided pharmaceutical compositions, comprising the described microvesicles.

Since non-autologous subcellular fractions may in some cases induce an immune reaction when administered to a subject, several approaches may be utilized according to the methods provided herein to reduce the likelihood of rejection of non-autologous mitochondria or other subcellular fractions. In some embodiments, these approaches include either suppressing the recipient immune system or encapsulating the subcellular fractions or mitochondria in immune-isolating, semipermeable membranes before transplantation.

In other embodiments, an immunosuppressive agent is present in the pharmaceutical composition.

One may, in various embodiments, administer the pharmaceutical composition in a systemic manner (as detailed herein). Alternatively, one may administer the pharmaceutical composition locally e.g., via injection of the pharmaceutical composition directly into an exposed or affected tissue region of a patient. In other embodiments, the subcellular fraction(s) (e.g., mitochondria or exosomes) are administered intravenously (IV), subcutaneously (SC), by the intraosseous route (e.g., by intraosseous infusion), or intraperitoneally (IP), each of which is considered a separate embodiment. In other embodiments, the subcellular fraction(s) (e.g. mitochondria or exosomes) are administered intracerebrally, by intracerebroventricular administration, intrathecally, or intranasally, each of which represents a separate embodiment. In other embodiments, the subcellular fraction(s) (e.g., mitochondria or exosomes) are administered intramuscularly; while in other embodiments, the subcellular fraction(s) (e.g., mitochondria or exosomes) are administered systemically. Intramuscular administration refers to administration into the muscle tissue of a subject; subcutaneous administration refers to administration just below the skin; intravenous administration refers to administration into a vein of a subject; intraosseous administration refers to administration directly into bone marrow; intraperitoneal administration refers to administration into the peritoneum; intracerebral administration refers to administration into the brain; intracerebroventricular administration refers to administration into the ventricles of the brain; intrathecal administration refers to administration into the subarachnoid space (which targets, in some embodiments, the cerebrospinal fluid [CSF]); and intranasal administration refers to administration into the nose, which is, in some embodiments, administration of a liquid suspension into the nose. In still other embodiments, the subcellular fraction(s) (e.g., mitochondria or exosomes) are administered intratracheally, by inhalation, or intranasally. In certain embodiments, lung-targeting routes of administration may utilize mitochondria encapsulated in liposomes or other barriers to reduce entrapment within the lungs.

In still other embodiments, the pharmaceutical composition is administered intralymphatically, for example as described in U.S. Pat. No. 8,679,834 in the name of Eleuterio Lombardo and Dirk Buscher, which is hereby incorporated by reference.

In other embodiments, for injection, the described fractions may be formulated in aqueous solutions, e.g., in physiologically compatible buffers such as Hank's solution, Ringer's solution, or physiological salt buffer, optionally in combination with cryopreservation agents.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals.

The data obtained from these in vitro and cell culture assays and animal studies can be used in formulating a range of dosage for use in human. The dosage may vary depending upon the dosage form employed and the route of administration utilized. The exact formulation, route of administration and dosage can be, in some embodiments, chosen by the individual physician in view of the patient's condition.

A typical human dosage of the described mitochondrial preparations ranges, in some embodiments, from about 1×10⁶-1×10¹¹ mitochondria. In other embodiments, a dose of 2×10⁶-5×10¹⁰, 5×10⁶-5×10¹⁰, 1×10⁷-5×10¹⁰, 2×10⁷-5×10¹⁰, 5×10⁷-5×10¹⁰, 1×10⁸-5×10¹⁰, 2×10⁸-5×10¹⁰, 5×10⁸-5×10¹⁰, 1×10⁹-5×10¹⁰, 2×10⁹-5×10¹⁰, 5×10⁹-5×10¹⁰, 2×10⁶-1×10¹⁰, 5×10⁶-1×10¹⁰, 1×10⁷-1×10¹⁰, 2×10⁷-1×10¹⁰, 5×10⁷-1×10¹⁰, 1×10⁸-1×10¹⁰, 2×10⁸-1×10¹⁰, 5×10⁸-1×10¹⁰, 1×10⁹-1×10¹⁰, 2×10⁹-1×10¹⁰, or 5×10⁹-1×10¹⁰ mitochondria are administered. A typical density of mitochondria in the described mitochondrial preparations ranges, in some embodiments, from 1×10⁵-5×10⁷; 2×10⁵-5×10⁷; 3×10⁵-5×10⁷; 5×10⁵-5×10⁷; 1×10⁶-5×10⁷; 2×10⁶-5×10⁷; 3×10⁶-5×10⁷; 5×10⁶-5×10⁷; 1×10⁵-2×10⁷; 2×10⁵-2×10⁷; 3×10⁵-2×10⁷; 5×10⁵-2×10⁷; 1×10⁶-2×10⁷; 2×10⁶-2×10⁷; 3×10⁶-2×10⁷; 5×10⁶-2×10⁷; 1×10⁵-1×10⁷; 2×10⁵-1×10⁷; 3×10⁵-1×10⁷; 5×10⁵-1×10⁷; 1×10⁶-1×10⁷; 2×10⁶-1×10⁷; 3×10⁶-1×10⁷; 5×10⁶-1×10⁷; or 7-10×10⁶ mitochondria particles/ml.

Depending on the severity and responsiveness of the condition to be treated, dosing can be of a single or, in other embodiments, a plurality of administrations, with a course of treatment lasting from several days to several weeks or, in other embodiments, until alleviation of the disease state is achieved.

Compositions including the described preparations formulated in a compatible pharmaceutical carrier may also be prepared, placed in an appropriate container, and labeled for treatment of an indicated condition.

The described compositions may, if desired, be packaged in a container that is accompanied by instructions for administration. The container may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In other embodiments, the described subcellular fraction(s) (e.g., mitochondria or exosomes), or in other embodiments mitochondria-enriched cellular fractions, are suitably formulated as a pharmaceutical composition which can be suitably packaged as an article of manufacture. Articles of manufacture may comprise a packaging material which includes a label describing a use in treating a disease or disorder or therapeutic indication mentioned herein. In other embodiments, a pharmaceutical agent is contained within the packaging material, wherein the pharmaceutical agent is effective for the treatment of a disorder or therapeutic indication that is mentioned herein. In some embodiments, the pharmaceutical composition is frozen.

It is clarified that each embodiment of the described subcellular fraction(s) (e.g., mitochondria or exosomes) and mitochondria-enriched cellular fractions may be freely combined with each embodiment relating to a therapeutic method or pharmaceutical composition.

### Subjects

In certain embodiments, the subject treated by the described methods and compositions is a human. In some embodiments, the subject is experiencing hematopoietic dysfunction, acute lung injury, cachexia, or mtDNA defects. Alternatively or in addition, the subject presents with pneumonia. In still other embodiments, the subject exhibits ischemia/reperfusion injury; stroke; neuronal dysfunctions, graft-versus-host disease (GvHD), or another inflammatory or autoimmune disease.

In certain embodiments, the subject is an elderly subject, for example a subject over 60, over 65, over 70, over 75, over 80, 60-85, 65-85, or 70-85 years in age; is a pediatric subject, for example a subject under 18, 15, 12, 10, 8, 6, 5, 4, 3, or 2 years, or under 18, 15, 12, 10, 8, 6, 5, 4, 3, 2, or 1 month in age; or is an adult subject, for example ages 18-60, 18-55, 18-50, 20-60, 20-55, 20-50, 20-45, 20-40, 20-35, 20-30, 25-60, 30-60, 40-60, or 50-60. In other embodiments, the subject may be an animal. In some embodiments, treated animals include domesticated animals and laboratory animals, e.g., non-mammals and mammals, for example non-human primates, rodents, pigs, dogs, and cats. In certain embodiments, the subject may be administered with additional therapeutic agents or cells.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including subcellular fractions. In another aspect, the kits and articles of manufacture may comprise a label, instructions, and packaging material, for example for treating a disorder or therapeutic indication mentioned herein.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

The cell expansion and harvest process consisted of 3 stages, followed by downstream processing steps: Stage 1, the intermediate cell stock (ICS) production; Stage 2, the thawing of the ICS and initial further culture steps; and Stage 3, bioreactor culture on Fibra-Cel® carriers. The downstream processing steps included cell harvest from flasks or bioreactor/s, cell concentration, washing, formulation, filling and cryopreservation. In general, culturing was performed in full DMEM, as described in Example 1 of International Patent Application WO 2016/098061, in the name of Esther Lukasiewicz Hagai et al., published on Jun. 23, 2016, which is incorporated herein by reference in its entirety. Placenta-derived cell populations containing over 90% maternal tissue-derived cells were prepared.

Osteogenesis and adipogenesis assays were performed on placental cells prepared as described in the previous paragraph and on BM adherent cells. In osteogenesis assays, over 50% of the BM cells underwent differentiation into osteocytes, while none of the placental-derived cells exhibited signs of osteogenic differentiation. In adipogenesis assays, over 50% of the BM-derived cells underwent differentiation into adipocytes. In contrast, none of the placental-derived cells exhibited morphological changes typical of adipocytes. These experiments were performed as described in Example 2 of WO 2016/098061, which is incorporated herein by reference.

### Example 2: Culture of Placental Cells in Serum-Free Medium

### Methods

The cell expansion and harvest process consisted of 3 stages, followed by downstream processing steps: Stage 1, the intermediate cell stock (ICS) production; Stage 2, the thawing of the ICS and initial further culture steps; and Stage 3, bioreactor culture on Fibra-Cel® carriers. The downstream processing steps included cell harvest from flasks or bioreactor/s, cell concentration, washing, formulation, filling and cryopreservation. The procedure included periodic testing of the growth medium for sterility and contamination, all as described in international patent application publ. no. WO 2019/239295, which is incorporated herein by reference. Bone marrow migration assays were also performed as described in WO 2019/239295.

### Results

Placental cells were extracted and expanded in serum-free (SF) medium for 3 passages. Cell characteristics of eight batches were assessed and were found to exhibit similar patterns of cell size and PDL (population doubling level since passage 1) as shown for a representative batch in Table 1. Cells also significantly enhanced hematopoiesis in a bone marrow migration (BMM) assay. Subsequent culture steps (ICS production, further 2D culturing, and bioreactor culturing) were also performed in SF medium.

TABLE 1
Characteristics of placental cells expanded in SF medium.

| | | | Total growth | cell size | |
|---|---|---|---|---|---|
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Osteocyte and Adipocyte Differentiation Assays

ASC were prepared as described in Example 1. BM adherent cells were obtained as described in WO 2016/098061 to Esther Lukasiewicz Hagai and Rachel Ofir, which is incorporated herein by reference in its entirety. Osteogenesis and adipogenesis assays were performed as described in Example 2 of WO 2016/098061. Incubation of BM-derived adherent cells in osteogenic induction medium resulted in differentiation of over 50% of the BM cells, as demonstrated by positive alizarin red staining. On the contrary, none of the placental-derived cells exhibited signs of osteogenic differentiation. Next, a modified osteogenic medium comprising Vitamin D and higher concentrations of dexamethasone was used. Over 50% of the BM cells underwent differentiation into osteocytes, while none of the placental-derived cells exhibited signs of osteogenic differentiation.

Adipocyte induction. Adipocyte differentiation of placenta- or BM-derived adherent cells in adipocyte induction medium resulted in differentiation of over 50% of the BM-derived cells, as demonstrated by positive oil red staining and by typical morphological changes (e.g., accumulation of oil droplets in the cytoplasm). In contrast, none of the placental-derived cells differentiated into adipocytes. Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 4: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat #A1007001) and the STEMPRO® Osteogenesis Differentiation Kit (GIBCO, Cat #A1007201), respectively.

### Results

Adipogenesis and Osteogenesis of placental cells grown in SRM (3 different batches) or in full DMEM were tested. In adipogenesis assays, BM-MSCs treated with differentiation medium stained positively with Oil Red O. By contrast, ⅔ of the SRM batches exhibited negligible staining, and the other SRM batch, as well as the full DMEM-grown cells, did not exhibit any staining at all, showing that they lacked significant adipogenic potential. In osteogenesis assays, BM-MSCs treated with differentiation medium stained positively with Alizarin Red S. By contrast, none of the placental cell batches grown in SRM or full DMEM exhibited staining, showing that they lacked significant osteogenic potential.

### Example 5: Characterization of Exosomes and Other Extracellular Vesicles from Cultured Placental Adherent Cells

Cultured placental adherent cells were expanded as described for Example 2. Spent bioreactor medium sample 2 was subjected to TFF (tangential flow filtration), resulting in 20-fold concentration, followed by 10 min, 300×G centrifugation (to remove intact cells); 10 min, 2000×G centrifugation (to remove dead cells); 30 min, and 10,000×G centrifugation (to remove cellular debris). Exosomes were isolated by ultracentrifugation for 70 min at 100,000-200,000×G which pelleted the exosomes. Exosomes were resuspended in a relatively small volume, and had the characteristics shown in Table 2, including a zeta potential indicative of high stability.

TABLE 2
Characteristics of exosomes isolated from placental cell medium.

| | Particle size | Concentration | Zeta |
|---|---|---|---|
| | (nm) | (particles/mL) | potential |
| Sample 1 | 179 | 1.98E+10 | +/−38 |
| Sample 2 | 197 | 9.11E+9 | +/−37.5 |

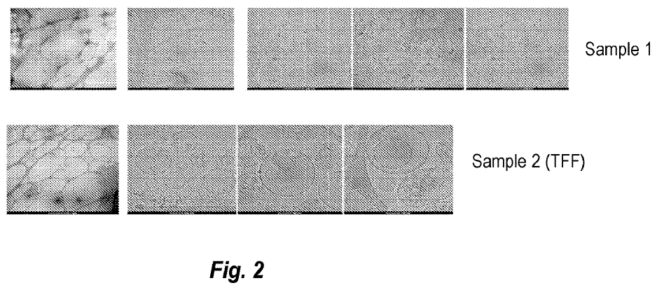

Cyro-TEM indicated a morphology consistent with exosomes, with a size peak of 170-22 nm. Electron microscopy indicated a heterogeneous size range (100-400 nm) and a characteristic lipid bilayer morphology (similar to liposomes) (FIG. 2). Identification of exosomes was further validated by MS identification of the exosome-specific markers CD63, CD81, CD9, and Annexins.

Mass spectrophotometry (MS) indicated the presence of 1391 total proteins, including 1221 common proteins, and 144 and 26 proteins found in only sample 1 or sample 2, respectively.

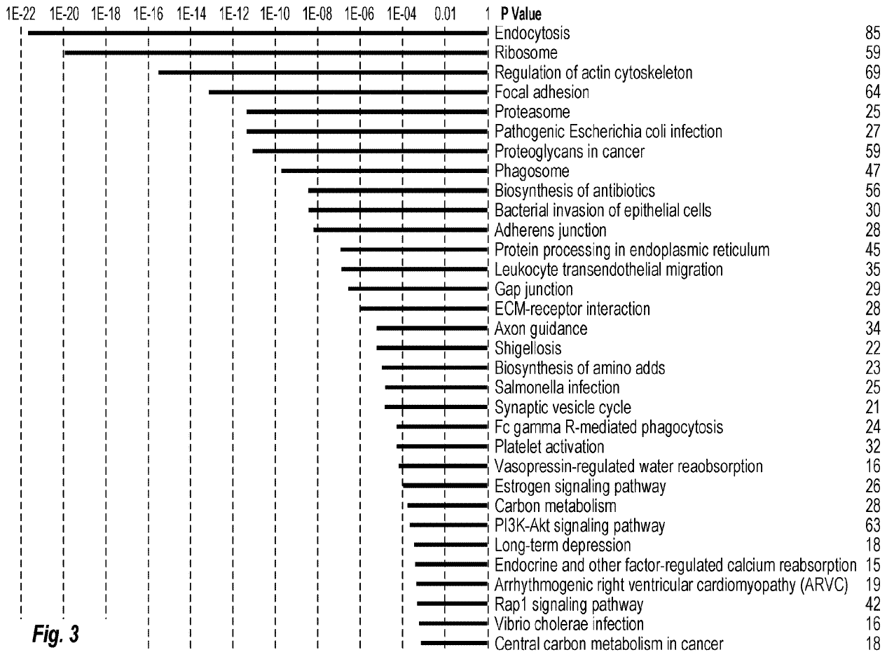

For sample 1, Kyoto Encyclopedia of Genes and Genomes (KEGG) analysis was used to identify biological pathways in which the identified proteins were enriched. KEGG does not consider relative or absolute abundance of detected proteins, but rather classifies/ordinates all proteins detected. Certain pathways were strikingly enriched, including endocytosis, ribosome, regulation of actin cytoskeleton, focal adhesion, proteosome, and phagosome (FIG. 3). GO-TERM enrichment analysis, which accounts also for abundance of the particular proteins, identified the following enriched cellular components, starting from most enriched: extracellular region part, extracellular organelle, extracellular vesicle, extracellular exosome, vesicle, extracellular space, plasma membrane, cell-substrate junction, cell-substrate adherens junction, and focal adhesion. Most enriched biological pathways were regulation of localization, regulated exocytosis, regulation of protein insertion into mitochondria membrane involved in apoptotic signaling, positive regulation of protein insertion into mitochondria membrane involved in apoptotic signaling, secretion, positive regulation of mitochondrial outer membrane permeability involved in apoptotic signaling, cell activation, exocytosis, and immune effector process. The most enriched individual proteins by MS were Serum albumin, Haptoglobin; Collagen alpha-3(VI) chain; Hemopexin; Neuroblast differentiation-associated protein (AHNAK); Filamin-A; Plectin; Actin, cytoplasmic 1; Myosin-9; Annexin A6; Afamin; Annexin A2; Aminopeptidase N; Moesin; Actin; Vimentin; Fibronectin; Integrin beta-1; Talin-1; Collagen alpha-1(VI) chain; Heat shock cognate 71 kDa protein; Filamin-B; Integrin alpha-2; Alpha actinin-1; Myoferlin; Collagen alpha-2(VI) chain; Alpha actinin-4; Ezrin; Brain acid soluble protein 1; Sodium/potassium-transporting ATPase subunit alpha-1; 5′-nucleotidase; Prolow-density lipoprotein receptor-related protein 1; Heat shock protein HSP 90-beta; Pyruvate kinase PKM; and Fatty acid synthase.

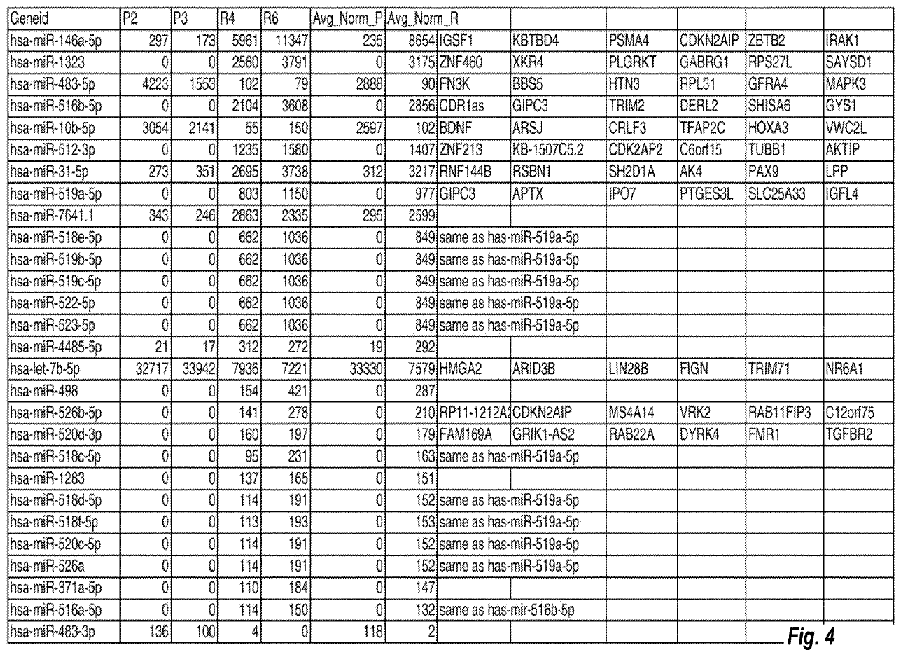

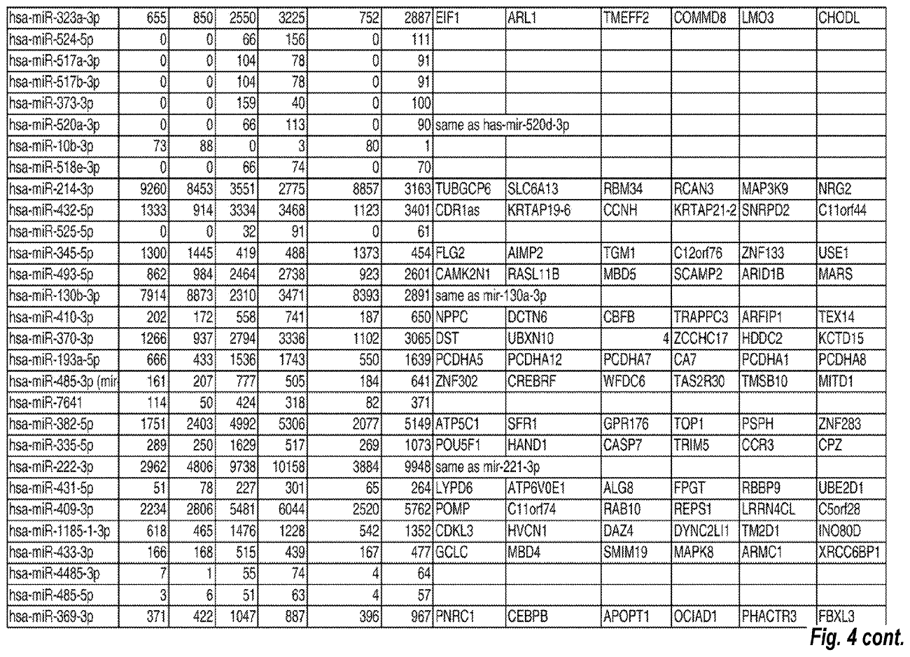

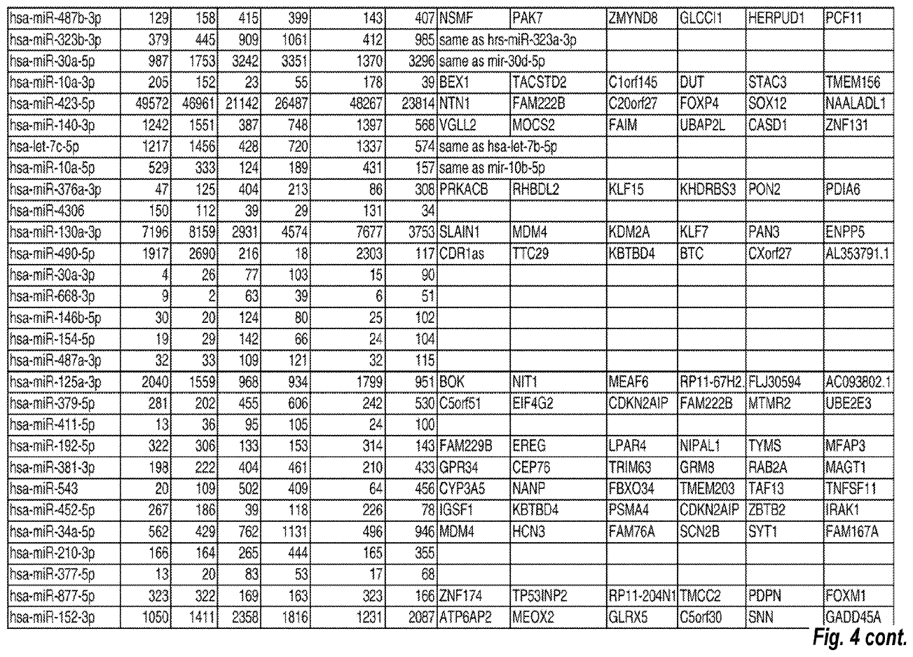

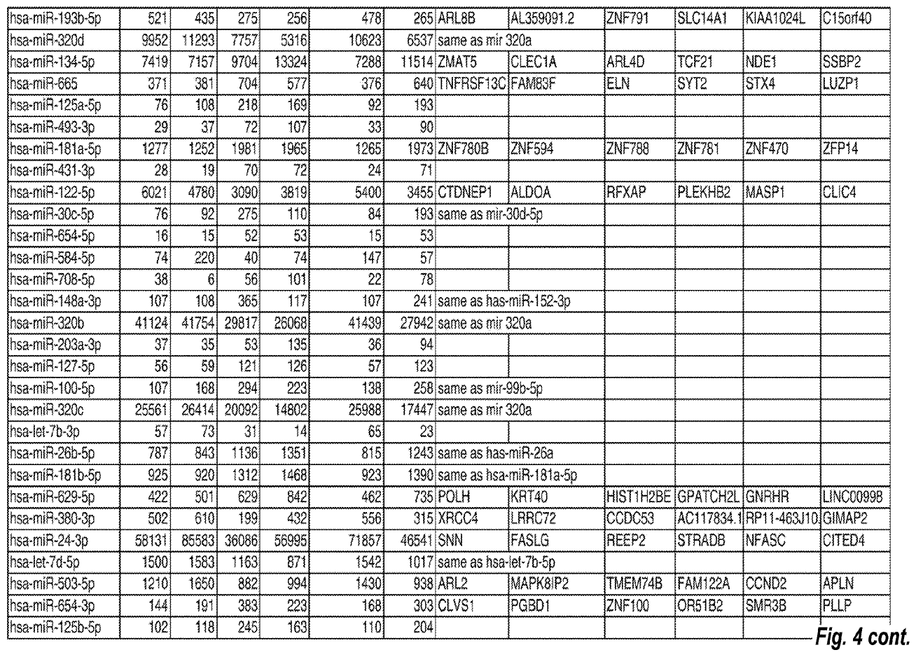

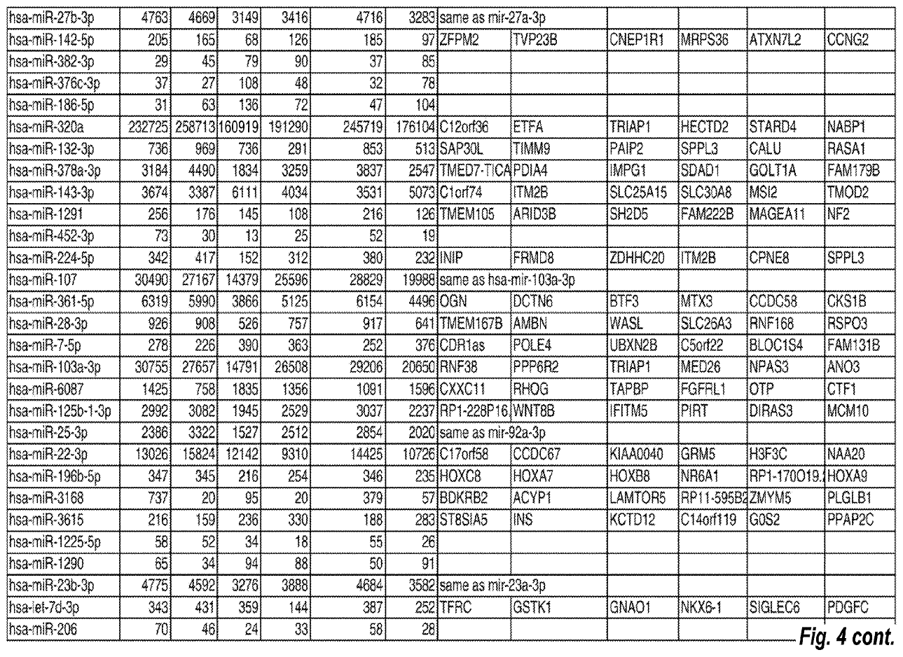

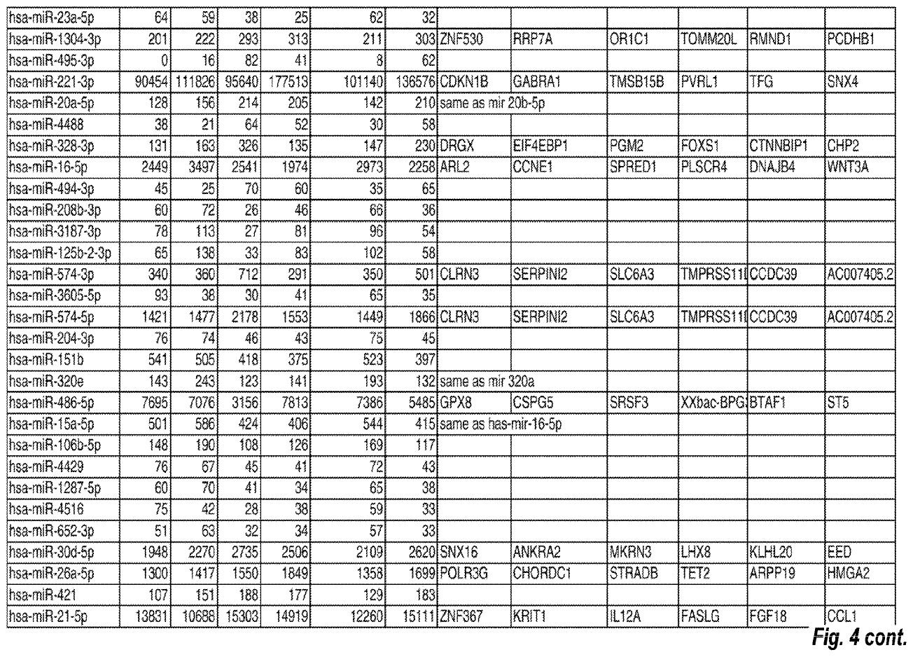

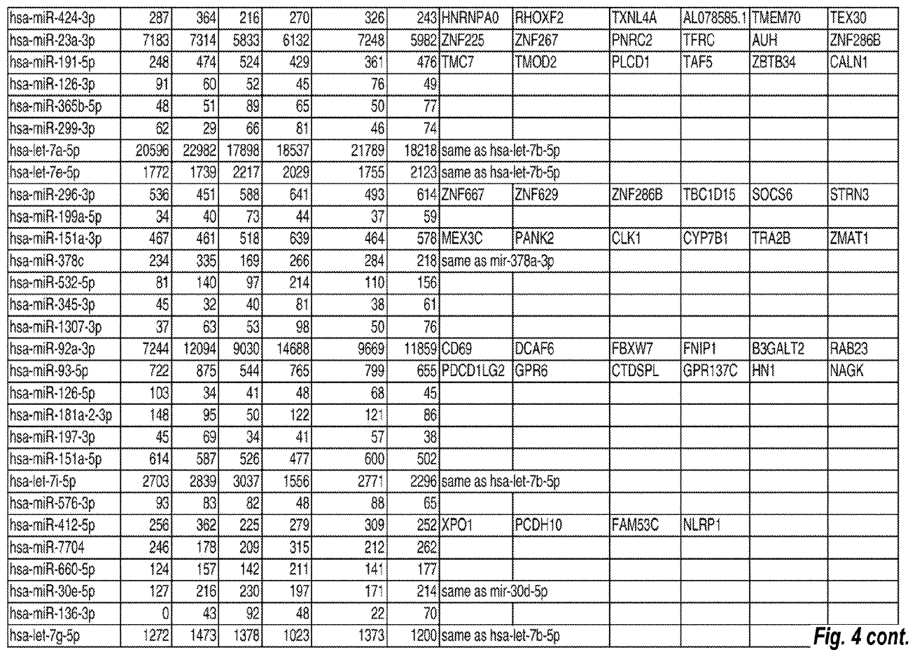

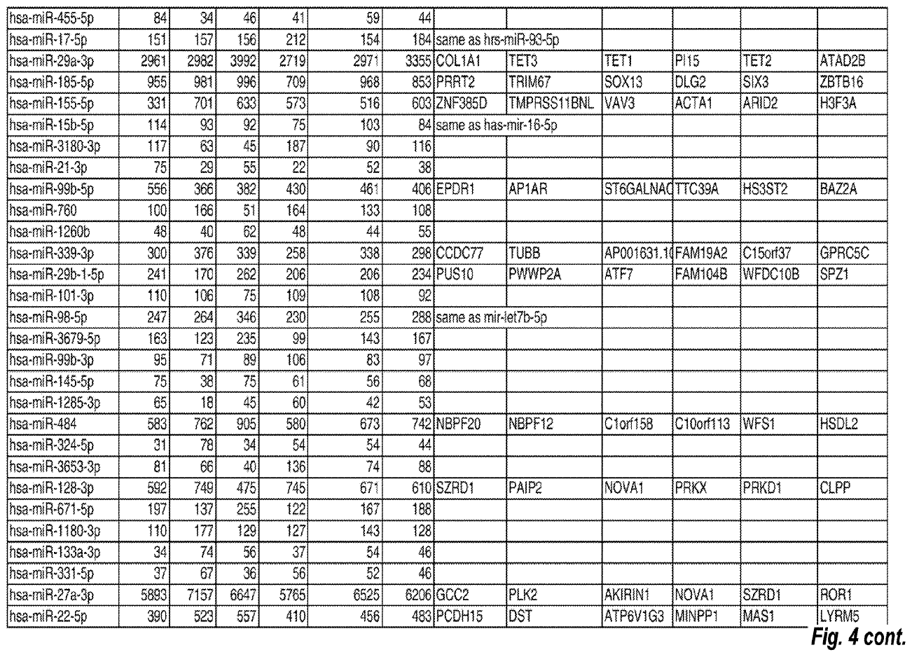

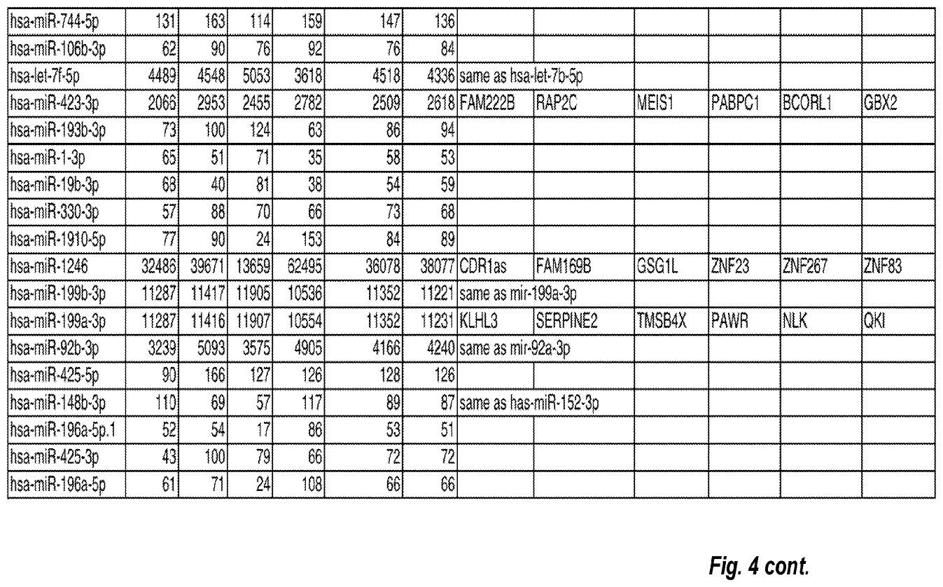

In another experiment, exosomes were isolated from 3 samples each of media from placental/maternal or placental/fetal cells using ExoQuickTC™. Protein and exosomes were quantitated using Qubit™ and CD63 ELISA, respectively (Table 3). RNA was isolated from the exosomes, using SeraMir™ Exosome RNA Purification Column kit, and small RNA concentration was determined using Agilent Bioanalyzer Small RNA Assay. NGS libraries were prepared using CleanTag® Small RNA Library Kit, cDNA libraries were amplified using PCR, and size selection (140-270 bp) was performed on a TBE polyacrylamide gel, followed by Next-Generation Sequencing. Identified siRNA are set forth in FIG. 4, together with Uniprot codes of representative proteins inhibited by each miRNA, indicating a portion of the biological activity of these placental subcellular fractions.

TABLE 3
Exosome content of maternal (1-3) and fetal
(4-6) placental cell preparations.

| | # Exosomes/25 μg | # Exosomes/ml | Small RNA |
|---|---|---|---|
| Name | protein | media | content |
| P1 | 7,888,000,000 | 4.33E+10 | 3.87 ng/μl |
| P2 | 7,708,000,000 | 4.24E+10 | 49.4 ng/μl |
| P3 | 8,088,000,000 | 4.44E+10 | 41.6 ng/μl |
| R4 | 7,215,500,000 | 1.19E+10 | 15.4 ng/μl |
| R5 | 7,695,500,000 | 6.67E+09 | 0.35 ng/μl |
| R6 | 6,940,500,000 | 2.86E+10 | 36.2 ng/μl |

### Example 6: Alleviation of Mitochondrial Dysfunction by Treatment with Placental ASC

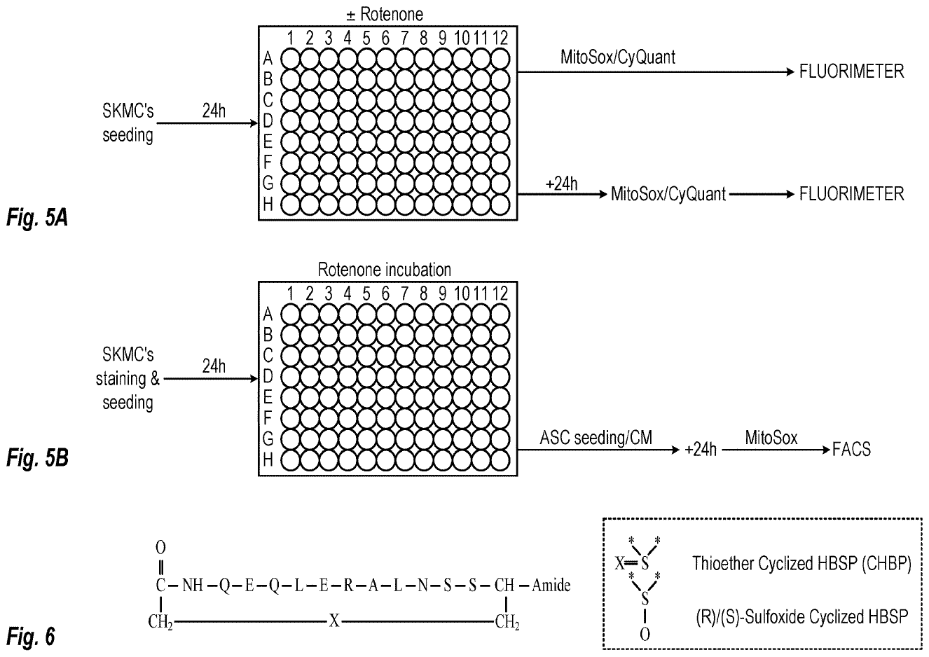

Skeletal muscle cells (SKMC) are seeded in multi-well plates and treated with rotenone to induce mitochondrial dysfunction. Mitochondria dysfunction is measured by MitoSox/CyQuant staining, followed by fluorescence measurement (FIG. 5A). Next, rescue of mitochondrial dysfunction by ASC or CM from ASC is assessed (FIG. 5B), followed by microscopic verification of mitochondrial transfer from ASC to SKMCs with mitochondrial dysfunction.

### Example 7: Treatment of Acute Lung Injury by Administration of Mitochondria from Placental ASC

Subjects with acute lung injury are treated with aerosolized formulations containing mitochondria from placental ASC or placental ASCs. Alleviation of lung injury is evidence of therapeutic efficacy.

### Example 8: Treatment of Muscle Wasting by Treatment with Mitochondria from Placental ASC

Subjects suffering from muscle wasting are treated systemically with pharmaceutical suspensions containing mitochondria from placental ASC or placental ASCs. Arrest or alleviation of muscle wasting is evidence of therapeutic efficacy.

### Example 9: Treatment of Hematopoietic Dysfunction by Administration of Autologous Cells Given Placental Mitochondria

Hematopoietic cells from subjects with hematopoietic dysfunction are treated ex-vivo with mitochondria from placental ASC, then are returned to the subject. Alternatively placental ASCs are administered inra-osseously and transfer mitochondria to BM hematopoietic stem cells. Alleviation of hematopoietic dysfunction is evidence of therapeutic efficacy.

### Example 10: Treatment of Hematopoietic Dysfunction by Administration of Allogeneic Cells Given Placental Mitochondria

Hematopoietic cells from healthy donors are treated ex-vivo with mitochondria from placental ASC, then are administered to HLA-matched subjects with hematopoietic dysfunction. Alleviation of hematopoietic dysfunction is evidence of therapeutic efficacy.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

1. A method of enhancing a cellular or a physiological function in a subject in need, comprising contacting the subject with
a. a pharmaceutical composition comprising a cellular fraction of a cultured placental adherent stromal cell (ASC); or
b. an enriched cell, wherein said enriched cell has been incubated with a composition comprising (i) a cultured placental adherent stromal cell (ASC); or (ii) a subcellular fraction of cultured placental ASC;
thereby enhancing a cellular or physiological function in the subject.

2. The method of claim 1, wherein said pharmaceutical composition is administered via intramuscularly, intravenously, subcutaneously, intraperitoneally, intradermally, intranasally, or by intracerebral injection, intracerebroventricular administration, or intrathecal administration.

3. The method of claim 1, wherein said cellular function is cellular aerobic respiration, and said subcellular fraction comprises a naked or an encapsulated mitochondrion.

4. The method of claim 1, wherein said subcellular fraction is a vesicular fraction.

5. The method of claim 1, wherein said subcellular fraction is selected from mitochondria, endoplasmic reticulum, Golgi apparatus, lysosomes, peroxisomes, exosomes, endosomes, ectosomes, microparticles, and microvesicles.

6. The method of claim 1, wherein said subcellular fraction has been prepared by a process comprising chemical or physical lysis of said placental ASC.

7. The method of claim 1, wherein incubating said cell with said composition to produce said enriched cell, comprises targeted introduction of the subcellular fraction or mitochondria into said enriched cell.

8. The method of claim 1, wherein said enriched cell is a stem cell.

9. The method of claim 8, wherein said stem cell is a hematopoietic stem cell or an induced pluripotent stem cell.

10. The method of claim 1, wherein the enriched cell is indicated for administration to a subject who exhibits disorder or disease selected from hematopoietic dysfunction, acute lung injury, cachexia, or mtDNA defects.

11. The method of claim 1, wherein said placental ASC express a marker selected from the group consisting of CD73, CD90, CD29 and CD 105, and/or wherein said placental ASC do not express a marker selected from the group consisting of CD3, CD4, CD11b, CD14, CD19, and CD34, and/or wherein said placental ASC do not express a marker selected from the group consisting of CD3, CD4, CD34, YCD39, and CD106.

12. The method of claim 1, wherein said placental ASC have been incubated on a 2D substrate and/or wherein said placental ASC have been incubated on a 3D substrate subsequent to incubating on a 2D substrate.

13. The method of claim 1, wherein said placental ASC have been incubated on a 3D substrate in a bioreactor.

14. A method of enhancing a physiological function in a subject in need, said method comprising contacting the subject with an enriched cell, wherein said enriched cell has been incubated with cultured placental adherent stromal cell (ASC), thereby enhancing a physiological function in said subject.

15. An enriched cell wherein said enriched cell has been contacted with a mitochondrion of a cultured placental adherent stromal cell (ASC).

16. A pharmaceutical composition comprising the enriched cell of claim 15.

17. The enriched cell of claim 15, wherein said enriched cell is used for enhancing aerobic respiration in a subject in need, upon contacting the subject with the enriched cell, wherein said enriched cell has been incubated with a composition comprising a mitochondrion of a cultured placental adherent stromal cell (ASC), thereby enhancing aerobic respiration in said subject.

18. The enriched cell of claim 17, wherein said composition has been prepared by a process comprising immuno-isolation of said mitochondria.

19. The enriched cell of claim 15, wherein the contact with the mitochondrion of the cultured placental ASC is performed ex vivo.

20. The enriched cell of claim 15, wherein the contact with the mitochondrion of the cultured placental ASC comprises coculturing the placental ASC with the enriched cell.
